(19) United States
(12) Patent Application Publication
(10) Pub. No.: US 2005/0182020 A1
(43) Pub. Date: Aug. 18, 2005

(54) Ceramide de novo synthesis-based therapeutic and prophylactic methods, and related articles of manufacture

(76) Inventors: Tilla S. Worgall, New York, NY (US); Richard J. Deckelbaum, Hastings-on-Hudson, NY (US)

Correspondence Address: John P. White;Cooper & Dunham LLp, 1185 Avenue of the Americas, New York, NY, 10036, US

(21) Appl. No.: 10/712,684

(22) Filed: Nov. 14, 2003

(51) Int. Cl. A61K 31/739; A61K 31/336; A61K 31/202; A61K 31/138; A61K 31/05

(52) U.S. Cl. 514/54; 514/560; 514/475; 514/651; 514/733

(57) ABSTRACT

Described is a method for decreasing the amount of mSREBP in a cell characterized by an elevated level of mSREBP comprising contacting the cell with an agent that specifically inhibits de novo synthesis of ceramide in the cell, thereby decreasing the amount of mSREBP in the cell. Also described are related methods and articles of manufacture.

This application claims priority of provisional application U.S. Ser. No. 60/425,354, filed Nov. 11, 2002, the contents of which are incorporated herein by reference.

The invention described herein was made with government support under NIH Grant T32DK07715. Accordingly, the United States government has certain rights in this invention.

Throughout this application, various references are cited. Disclosure of these references in their entirety is hereby incorporated by reference into this application to more fully describe the state of the art to which this invention pertains.

### BACKGROUND OF THE INVENTION

SREBP and Ceramide

The sterol regulatory element binding-proteins (SREBPs) are pivotal transcription factors of genes of cholesterol, fatty acid and carbohydrate metabolism. Precursor SREBP (pSREBP) is located in the endoplasmic reticulum, where it is bound at the C-terminal end to the SREBP cleavage activating protein (SCAP). In sterol depletion, both proteins are translocated by vesicular trafficking to the Golgi apparatus (1, 2). Sequential cleavage by two proteases, site-1-protease (S1P) and site-2-protease (S2P), releases the transcriptionally active mature SREBP (mSREBP). In the nucleus, mSREBP binds to sterol regulatory elements (SRE), cis-acting elements in the promoters of genes of cholesterol and fatty acid synthesis (3).

Cholesterol and unsaturated fatty acids are known regulators of transcriptional and post-transcriptional processing of SREBP. Of interest, there is further evidence of cholesterol-independent regulation of SREBP (4-8). Drosophila melanogaster SREBP is only regulated by palmitic acid but not by cholesterol or unsaturated fatty acids (9). It has recently been reported that unsaturated fatty acid-mediated decreases in SRE-mediated gene transcription are linked to cellular sphingolipid metabolism (10). Ceramide, a metabolite of sphingomyelin hydrolysis, also regulates levels of the mature, transcriptionally active SREBP. Importantly, ceramide decreases SRE-mediated gene transcription in the presence of inhibitors of intracellular cholesterol trafficking, suggesting a cholesterol-independent regulatory effect (10).

Ceramide is a hydrophobic molecule with a slow interbilayer movement and has multiple roles ranging from lipid second messenger to the induction of apoptosis, cell growth and differentiation (11, 12). Cellular ceramide levels are generated either de novo by serine-palmitoyl transferase from serine and palmitoyl-CoA or through a recycling pathway of sphingolipid hydrolysis. It has been suggested that rapidly dividing cells utilize the de novo pathway of sphingolipid synthesis, whereas slowly dividing cells predominantly synthesize ceramide and sphingolipids from sphingoid bases salvaged from the hydrolytic pathway (13). Increased endogenous sphingolipids, molecules derived from ceramide, alter the intracellular distribution of cholesterol and result in defective sorting and transport of sphingolipids (14). Ceramide also has a role in intracellular protein trafficking. Ceramide can inhibit coated vesicle formation and exocytosis in CHO cells (15), inhibit intracellular trafficking of the VSVG virus protein through the Golgi apparatus (16) and can modulate endocytosis in mammalian cells (17). In yeast, ongoing ceramide de novo synthesis is critical in the vesicular ER to Golgi transport of GPI-anchored proteins (18-20).

Heriditary Sensory Neuropathy and Niemann Pick Disease

Heriditary Sensory Neuropathy type 1 (HSN1) is the most common hereditary disorder of peripheral sensory neurons. HSN1 is an autosomal dominant progressive degeneration of dorsal root ganglia and motor neurons with onset in the second or third decades. Initial symptoms are sensory loss in the feet followed by distal muscle wasting and weakness. Loss of pain sensation leads to chronic skin ulcera and possible distal amputations. Two independent groups demonstrated that mutations in serine-palmiltoyl transferase long chain base subunit-1 (SPTLC1) causes hereditary sensory neuropathy type 1 [26, 27]. Both groups differ in their experimental findings with respect to the effects of the mutations on the activity of serine-palmitoyl transferase (SPT). SPT is the rate limiting enzyme in de-novo ceramide synthesis. Dawkins & Nicholson show increased de novo synthesis of phosphatiylethanolamine, phosphatiylserine and glucosylceramide compared to controls (incubation with radioactive tracer ³H-serine for 4 h) in human lymphoblasts [1]. Bejaoui and Hanada show decreased sphingolipid synthesis (³H-serine as a radioactive tracer/2.5 h incubation) [28]. Transformed human HSN1 lymphoblasts (HSN 4561) and controls (HSN 4513) were received from K. Bejaoui to investigate the regulation of SREBP and SRE-mediated lipid metabolism in these cells. The HSN1 lymphoblasts (4561) have the C 133 Y mutation.

Niemann Pick Disease is an autosomal recessive lysosomal storage disease. Niemann Pick Disease is defined by accumulation of cholesterol and sphingolipids, and presence of “foam cells” in tissues and bone marrow. The disease is also defined by mutations in the acid sphingomyelinase gene. There are two types distinguished by the amount of acid sphingomyelinase activity. Activity of acid sphingomyelinase below 5% (Type A) results in severe neurological disease and death by an early age (i.e., 3-4 years). An activity above 10% (Type B) is sufficient to protect the central nervous system from devastating disease. Individuals with Type B have a variable phenotype, are neurologically intact, have pulmonary infiltration and live to adulthood.

### SUMMARY OF THE INVENTION

This invention provides a method for decreasing the amount of mSREBP in a cell characterized by an elevated level of mSREBP comprising contacting the cell with an agent that specifically inhibits de novo synthesis of ceramide in the cell, thereby decreasing the amount of mSREBP in the cell.

This invention also provides a method for decreasing cholesterol synthesis in a cell characterized by an elevated level of mSREBP comprising contacting the cell with an agent that specifically inhibits de novo synthesis of ceramide in the cell, thereby decreasing cholesterol synthesis in the cell.

This invention also provides a method for decreasing fatty acid synthesis in a cell characterized by an elevated level of mSREBP comprising contacting the cell with an agent that specifically inhibits de novo synthesis of ceramide in the cell, thereby decreasing fatty acid synthesis in the cell.

This invention also provides a method for decreasing triglyceride synthesis in a cell characterized by an elevated level of mSREBP comprising contacting the cell with an agent that specifically inhibits de novo synthesis of ceramide in the cell, thereby decreasing triglyceride synthesis in the cell.

This invention further provides a method for increasing the amount of mSREBP in a cell comprising contacting the cell with an agent that specifically increases de novo synthesis of ceramide in the cell, thereby increasing the amount of mSREBP in the cell.

This invention also provides a method for treating a subject afflicted with a disorder characterized by an elevated level of mSREBP in the subject's cells comprising administering to the subject a therapeutically effective amount of an agent that specifically inhibits de novo synthesis of ceramide in the subject's cells, thereby treating the subject.

This invention also provides a method for treating a subject afflicted with a disorder characterized by increased ceramide synthesis in the subject's cells comprising administering to the subject a therapeutically effective amount of an agent that specifically inhibits de novo synthesis of ceramide in the subject's cells, thereby treating the subject.

This invention also provides a method for treating a subject afflicted with an elevated cholesterol level comprising administering to the subject a therapeutically effective amount of an agent that specifically inhibits de novo synthesis of ceramide in the subject's cells, thereby treating the subject.

This invention also provides a method for treating a subject afflicted with an elevated fatty acid level comprising administering to the subject a therapeutically effective amount of an agent that specifically inhibits de novo synthesis of ceramide in the subject's cells, thereby treating the subject.

This invention also provides a method for treating a subject afflicted with an elevated triglyceride level comprising administering to the subject a therapeutically effective amount of an agent that specifically inhibits de novo synthesis of ceramide in the subject's cells, thereby treating the subject.

This invention also provides a method for inhibiting in a subject the onset of a disorder characterized by an elevated level of mSREBP in the subject's cells comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by increased ceramide synthesis in the subject's cells comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by an elevated cholesterol level in the subject comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by an elevated fatty acid level in the subject comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by an elevated triglyceride level in the subject comprising administering to the subject a prophylactically effective amount of an agent that specifically inhibits de novo synthesis of ceramide in the subject's cells, thereby inhibiting the onset of the disorder.

This invention also provides a method for increasing the amount of mSREBP in the cells of a non-human subject comprising administering to the subject an effective amount of an agent that specifically increases de novo synthesis of ceramide in the subject's cells, thereby increasing the amount of mSREBP in the subject's cells.

This invention also provides an article of manufacture comprising a packaging material having therein an agent that specifically inhibits de novo synthesis of ceramide in a cell, and a label indicating a use for the agent in treating or inhibiting the onset of a disorder in a subject, which disorder is characterized by an elevated level of mSREBP in the subject's cells.

This invention also provides an article of manufacture comprising a packaging material having therein an agent that specifically inhibits de novo synthesis of ceramide in a cell, and a label indicating a use for treating or inhibiting the onset of an elevated cholesterol level in a subject.

This invention also provides an article of manufacture comprising a packaging material having therein an agent that specifically inhibits de novo synthesis of ceramide in a cell, and a label indicating a use for treating or inhibiting the onset of an elevated fatty acid level in a subject.

This invention also provides an article of manufacture comprising a packaging material having therein an agent that specifically inhibits de novo synthesis of ceramide in a cell, and a label indicating a use for treating or inhibiting the onset of an elevated triglyceride level in a subject.

This invention also provides a method for determining whether an agent decreases de novo synthesis of ceramide in a cell, which method comprises the steps of (a) contacting the cell with the agent under suitable conditions; (b) determining the amount of de novo synthesis of ceramide in the cell after a suitable period of time; and (c) comparing the amount of de novo synthesis of ceramide determined in step (b) with the amount of de novo synthesis of ceramide in a cell in the absence of the agent, a lower amount of de novo synthesis of ceramide in the cell contacted with the agent indicating that the agent decreases the amount of de novo synthesis of ceramide in the cell.

Finally, this invention provides a method for determining whether an agent increases de novo synthesis of ceramide in a cell, which method comprises the steps of (a) contacting the cell with the agent under suitable conditions; (b) determining the amount of de novo synthesis of ceramide in the cell after a suitable period of time; and (c) comparing the amount of de novo synthesis of ceramide determined in step (b) with the amount of de novo synthesis of ceramide in a cell in the absence of the agent, a greater amount of de novo synthesis of ceramide in the cell contacted with the agent indicating that the agent increases the amount of de novo synthesis of ceramide in the cell.

### BRIEF DESCRIPTION OF THE FIGURES

FIG. 1. Exogenous and endogenous ceramide and dihydroceramide decreases incorporation of ³H-serine into de novo synthesized ceramide. On day 1, CHO cells were plated at 80% confluency in 35-mm dishes in triplicate. On day 2, cells were treated for 8 h either with control conditions (1% BSA) (negative control) and myriocin (1 μM) (positive control), C6-ceramide (20 μM) (C6-Cer), C8-ceramide (20 μM) (C8-Cer), dihydro-C6-ceramide (20 μM) (DHC6-Cer), D-MAPP (30 μM), PPMP (20 μM) or NB-DNJ (40 μM) After 6.5 h₁ ³H-serine (1 μl/ml) was added to each condition to measure incorporation of label into de novo synthesized ceramide. At 8 h, lipids were extracted using chloroform/methanol/0.1 N HCl followed by alkaline hydrolysis. Lipids were dried under N₂ and separated by TLC (methanol/chloroform/0.22% aqueous CaCl₂; 60:35:8 v/v). Ceramide spots were cut out from the TLC plate and radioactivity was determined. Data are expressed as dpm/protein and represent the average of four separate experiments, each performed in triplicate. All experimental conditions (except NB-DNJ, which does not increase endogenous ceramide levels) significantly decreased ceramide-associated ³H-serine compared to control (p<0.05), regardless of whether ceramide levels were increased exogenously (C6- and C8-ceramide) or endogenously through inhibition of ceramidase (D-MAPP) or glucosylceramide synthase (PPMP).

FIG. 2. C6-ceramide increases pSREBP levels. On day 1, CHO cells were plated in regular growth medium. On day 2, cells were incubated for 4 and 8 h with 1% BSA (control) or in the presence of C6-ceramide (20 μM). Whole cell extracts (30 μg protein) were loaded on a 4-14% continuous gradient SDS-PAGE gel. P denotes the precursor (125 kd) form of SREBP-1 in a representative experiment. C6 ceramide increases the precursor form of SREBP-1 and decreases the mature form of SREBP-1. The blot was then stripped and probed with an antibody against actin to demonstrate equal loading of the samples. The blot is representative of experiments carried out with ceramide analogues of different chain length. Densitometric results were obtained by analyzing pixels/inch (corrected for actin) and expressed relative to control.

FIG. 3. Inhibition of ceramide synthesis and SRE-mediated gene transcription. (A) Decreased ceramide synthesis correlates with decreased levels of SRE-mediated gene transcription and mSREBP. CHO cells stably transfected with an SRE-promoter construct linked to the luciferase reporter gene were incubated for 8 h in the presence of control conditions (1% BSA), cholesterol/25-OH cholesterol (10 μg/ml/1 μg/ml), myriocin (1 μM), cycloserine (500 mM), fumonisin B1 (20 μM), PPMP (20 μM) or NB-DNJ (negative control) (40 μM). After cell lysis, luciferase activity was analyzed, and expressed as a ratio of protein content. Data represent the average (±S.D.) of at least 4 different experiments, each performed in triplicate. Compared to control, all conditions except NB-DNJ significantly reduce luciferase expression (p<0.05) measured as relative light units (RLU). (B) Myriocin dose-dependently decreases SRE-mediated gene transcription. CHO cells stably transfected with an SRE-promoter construct linked to the luciferase reporter gene were incubated for 8 h in the presence of control conditions (1% BSA) or increasing levels of myriocin (0.25-1 μM). After cell lysis, luciferase activity was analyzed, expressed as a ratio of protein content. Data represent the average (±S.D.) of at least 3 different experiments, each performed in triplicate. Compared to control, all conditions significantly reduce luciferase expression (p<0.05) measured as relative light units (RLU).

FIG. 4. Increased ceramide synthesis and SRE-mediated gene transcription. (A) Increased ceramide de novo synthesis correlates with increased SRE-mediated gene transcription. On day 1, CHO cells stably transfected with an SRE-promoter construct linked to the luciferase reporter gene were plated at 80% confluency. On day 2, cells were incubated for 8 h in the presence of DMS, an inhibitor or sphingosine-1-P kinase (1.5-5 μM) or with sphingosine (1.5 μM). Cells were harvested, lysed and analyzed for luciferase activity (measured in relative light units, RLU) and protein content. Data are expressed as percentage of control and represent the average (±S.D.) of three different experiments, each performed in triplicate. Inset: Western blot analysis: On day 1, CHO cells were plated in regular growth medium. On day 2, cells were incubated for 8 h with control medium (1% BSA) or DMS (5 μM). Whole cell extracts (30 μg protein) were loaded on a 4-14% continuous gradient SDS-PAGE gel. P and M denote the precursor (125 kd) and mature (68 kd) form of SREBP-1 in a representative experiment. (B) DMS increases ceramide de novo synthesis. CHO cells were treated for 3.5 h either with control conditions (1% BSA) or DMS (2.5-5 μM). Then, ³H-sphingosine (1 μl/ml) was added to each condition to measure incorporation of label into de novo synthesized ceramide for 1.5 h. After 5 h, lipids were extracted using chloroform/methanol/0.1 N HCl followed by alkaline hydrolysis. Lipids were dried under N₂ and separated by TLC (methanol/chloroform/0.22% aqueous CaCl₂; 60:35:8 v/v). Ceramide spots were cut out from the TLC plate and radioactivity was determined. Data are expressed as dpm/protein and represent a typical experiment.

FIG. 5. LY-B cells fail to recover SRE-mediated gene transcription in cholesterol depletion but recover SRE-mediated gene transcription in the presence of DMS. On day 1, LY-B cells (CHO cells that are mutated in the LCB1 subunit of serine-palmitoyl transferase and do not synthesize ceramide de novo) and control CHO cells were plated in regular growth medium. Both cell lines have been transfected to stably express an SRE-promoter construct linked to the luciferase reporter gene. On day 2, cells were incubated for 16 h with 1% BSA (control) or in the presence of cholesterol (10 μg/ml) and 25-OH cholesterol (1 μg/ml) to decrease SRE-mediated gene transcription. Then, incubation medium was switched to 1% BSA or DMS (5 μM) for 6 h. After cell lysis, luciferase activity was analyzed, expressed as a ratio of protein content. Data represent the average (±S.D.) of at least 4 different experiments, each performed in triplicate. After 16 h, cholesterol decreases SRE-mediated gene transcription significantly stronger in LY-B cells (white bars) compared to control cells (black bars) (p<0.05). Incubation for 6 h in the presence of 1% BSA fails to increase SRE-mediated gene transcription in LY-B cells but significantly increases SRE-mediated gene transcription in control cells (p<0.05). Incubation with DMS (5 μM) significantly (p<0.05) increases SRE-mediated gene transcription in LY-B and control cells.

FIG. 6. Myriocin decreases levels of HMG-CoA synthase mRNA. CHO cells were incubated for 8 h with control medium (1% BSA) or myriocin (1 μM). 30 μg of total RNA were loaded per lane, electrophoresed on a 1.2% agarose/formaldehyde gel and transferred to a nylon membrane. The membrane was hybridized with ³²P-labeled probes for HMG-CoA synthase and glyceraldehyde-3-phosphate dehydrogenase (GAPDH) as outlined under “Experimental Details.” Lane 1: Control; lane 2: Myriocin (1 μM). The level of HMG-CoA synthase mRNA relative to control values was calculated after quantitative phosphorimager analysis and correction of loading differences determined by the glyceraldehyde-3-phosphate dehydrogenase signal.

FIG. 7. Ceramide de novo synthesis pathway.

FIG. 8. Ceramide catabolism pathway.

FIG. 9. Ceramide synthesis is increased in HSN cells.

FIG. 10. Increased SRE-mediated gene transcription in HSN1 cells (HSN 4561). Cells were transfected with an SRE-element containing promoter-reporter gene. This construct is identical to the promoter-reporter gene used in all previous studies. In order to achieve transfection in lymphoblasts, this promoter construct was subcloned into an adenovirus vector. Cells (control lymphoblasts and HSN1 lymphoblasts) were transfected for 3 h with this adenovirus vector and left in growth medium overnight. Medium was then switched to experimental medium containing 1% fatty acid free BSA (control conditions) or cholesterol/25-OH cholesterol (10 ug/ml/1 ug/ml) or oleic acid. After 7 h the luciferase activity which reflects binding of SREBP to the SRE-promoter element was measured. Because adenovirus mediated transfections are transient transfections the necessity for a transfection control arises. The adenovirus vector contains a β-gal control gene that is not regulated by experimental conditions and serves as a transfection control (standard practice). Results are expressed as a fraction of luciferase/β-gal and show significantly increased SRE-mediated gene expression in HSN 1 cells, confirming the first hypothesis. Cholesterol is very efficient in decreasing SRE-mediated gene transcription in HSN 1 (4561). Oleic acid does not decrease SRE-mediated gene transcription in either controls or HSN 1 (4561).

FIG. 11. Pathway demonstrating role of mevalonate and fatty acids in cholesterol and cholesterylester synthesis.

FIG. 12. Increased free cholesterol synthesis in HSN cells. In order to investigate whether increased SRE-mediated gene transcription also results in increased cholesterol synthesis cells were incubated with radioactive mevalonate, a precursor which represents a committed step in cholesterol synthesis (see FIG. 11). Control and HSN1 cells were incubated for 7 h and 18 h in the presence of trace amounts of ³H-mevalonate. Lipids were extracted and separated by thin liquid chromatography (TLC). Radioactive counts in the cholesterol fraction were analyzed by liquid scintillation counting. Results demonstrate that significantly more counts accumulate within 18 h in the cholesterol fraction in HSN1 cells (white bars). These data confirm increased cholesterol synthesis in HSN1 cells.

FIG. 13. Increased cholesteryl ester synthesis in HSN cells only from mevalonate and not from oleate. To evaluate the origin of cholesterylester synthesis, two different radioactive tracers were used over 4 h: ³H-mevalonate, to measure de novo synthesized cholesterol as a source of cholesterylester and ³H-oleate to include esterification of de novo synthesized and preexistent cholesterol. Preliminary data suggest that cholesterylester formation occurs preferentially from de novo synthesized cholesterol.

FIG. 14. Oleic acid increases HSN 4561 synthesis of high levels of free cholesterol but not cholesteryl ester. Cells were incubated for 4 h with ³H-mevalonate and grown in the presence or absence of 0.3 mM oleic acid. Results show that oleate stimulates the synthesis of cholesterol but not of cholesterylester. This data supports the hypothesis that the synthesis of cholesterol is altered and increased in HSN1 cells.

FIG. 15. Increased levels of free cholesterol in HSN 4561 (affected) cells. Measurement of free cholesterol and cholesteryl ester mass was carried out using gas chromatography (GC) in order to demonstrate that increased synthesis results in increased accumulation of mass. Cells were not specially treated but harvested straight from regular growth medium, washed and then lipids were extracted. GC analysis confirms increased cholesterol levels in HSN1 cells.

FIG. 16. Incubation with mevastatin decreases free cholesterol synthesis in HSN cells as well as in controls. Cells were treated for 16 h in the presence of ³H-mevalonate to assess cholesterol de novo synthesis and several inhibitors of either cholesterol or ceramide synthesis (Mevastatin=Statin), C6 ceramide (previously shown by us to decreased SREBP levels and SRE-mediated gene transcription), cycloserine (inhibitor of serine-palmiltoyl transferase), cholesterol (classical inhibitor of de novo synthesis by negative feedback). Results demonstrate that HSN1 cells (4561) are equally sensitive to statins in order to decrease cholesterol de novo synthesis and are more sensitive to C6 ceramide and cholesterol than control cells.

FIG. 17A-D. Controls (4513; A and B) and HSN1 cells (4561; C and D) were incubated with either BSA (control) or oleate (0.3 mM) in order to assess morphology. Cells were plated on glass slides and stained with ‘quick diff’ (not a lipid stain). HSN 1 cells (4561) show significant cytoplasmic inclusions. The nature of these inclusions is defined using Filippin, Nile Red and Oil Red O staining (data currently not available).

FIGS. 18A and B. Experiments carried out to investigate whether cells undergo cell death/apoptosis. Cells were incubated for 16 h in the presence of different experimental conditions and positive controls (staurosporin for caspase assay, triton x 1 uM for LDH assay). (A) Data show that HSN1 cells do not increase caspases 3/7 more than controls. Caspases 3/7 measure apoptotic (cell death) pathways. (B) The LDH assay measured cell toxicity. Cells were incubated for 20 h. HSN1 cells (4561) showed higher cell toxicities to experimental conditions than controls. Differences are not considered significant compared to positive controls (triton x). These data verify that the lymphoblasts survive the treatment conditions and that apoptosis or cell death is not induced. The data do not rule out a cytotoxic effect on small unmyelinated nerve fibers.

FIG. 19. Ceramide synthesis is increased in NPA fibroblasts. Fibroblasts from different control individuals and from two different cell lines derived from Niemann Pick Disease Type A were incubated for 4 h in the presence of ³H-serine to measure de novo ceramide synthesis after having been incubated overnight in either control medium (1% BSA) or in the presence of cholesterol or C8-Ceramide. Lipid extraction and separation were carried out. Results demonstrate that de-novo synthesis of ceramide is increased in NPA cells (black bars). Cholesterol does not significantly decrease ceramide de novo synthesis (gray bars). C8-Ceramide significantly decreases ceramide de novo synthesis (white bars).

FIG. 20. SRE-mediated gene transcription is increased in NPA fibroblasts. The hypothesis was evaluated that increased ceramide de novo synthesis affects SREBP and SRE-mediated gene transcription in Niemann Pick Type A cells. Cells were transfected with an SRE-promoter construct (using adenoviral transfection as above in HSN cells). Results show that SRE-mediated gene transcription is significantly increased in NPA cells compared to controls. Addition of cholesterol decreases SRE-mediated gene transcription and this process is also reversible. Reversibility is demonstrated by the third set of bars: When cells are first incubated in cholesterol and then the medium is switched to BSA (cholesterol depletion) SRE-mediated gene transcription increases again in controls as well as in NPA cells.

FIG. 21. Cholesterol synthesis is increased in NPA fibroblasts. Two different controls and two different NPA cells are incubated for 16 h in the presence of ³H-mevalonate in the presence of control condition (BSA; black bars), cholesterol (gray bars) or C8 Ceramide (white bars). Label incorporation into free cholesterol is measured.

FIG. 22. Cholesterol mass is measured by gas chromatography and shown to be increased. This data shows that increased synthesis results in increased mass.

FIG. 23. Triglyceride synthesis is increased in NPA cells. SREBP also regulates pathways of triglyceride synthesis. Therefore triglyceride synthesis by measurement of ³H-oleate incorporation was measured and shown to be increased in NPA cells.

### DETAILED DESCRIPTION OF THE INVENTION

Definitions

As used in this application, except as otherwise expressly provided herein, each of the following terms shall have the meaning set forth below.

“Administering” shall mean delivering in a manner which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. Administering can also be performed, for example, once, a plurality of times, and/or over one or more extended periods.

“Agent” shall mean any chemical entity, including, without limitation, a small molecule, a glycomer, a protein, an antibody, a lectin, a nucleic acid and any combination thereof.

“Cells” include, without limitation, normal, abnormal and transformed cells, either isolated from a subject or an established cell line, and are exemplified by neurons, epithelial cells, muscle cells, blood cells, immune cells, stem cells, hepatocytes, adipocytes, osteocytes, endothelial cells and blast cells. In the preferred embodiment of this invention, the cells are hepatocytes or adipocytes.

“Pharmaceutically acceptable carriers” are well known to those skilled in the art and include, but are not limited to, 0.01-0.1 M and preferably 0.05 M phosphate buffer or 0.8% saline.

Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Prophylactically effective amount” means an amount sufficient to inhibit the onset of a disorder in a subject. Simple titration experiments can readily be performed by one of ordinary skill to determine such amount.

“Specifically inhibiting” ceramide de novo synthesis includes, without limitation, (i) inhibiting ceramide de novo synthesis without inhibiting all other synthetic pathways, (ii) inhibiting ceramide de novo synthesis more than most or any other synthetic pathway, and/or (iii) inhibiting ceramide de novo synthesis without inhibiting any other synthetic pathway.

“Subject” shall mean any animal, such as a mammal or a bird, including, without limitation, a cow, a horse, a sheep, a pig, a dog, a cat, a rodent such as a mouse, rat or hamster, a chicken and a primate. In the preferred embodiment, the subject is a human.

“Therapeutically effective amount” means an amount sufficient to treat a subject. Simple titration experiments can readily be performed by one of ordinary skill to determine such amount.

“Treating” means either slowing, stopping or reversing the progression of a disorder. As used herein, “treating” also means the amelioration of symptoms associated with the disorder.

### Embodiments of the Invention

This invention provides a method for decreasing the amount of mSREBP in a cell characterized by an elevated level of mSREBP comprising contacting the cell with an agent that specifically inhibits de novo synthesis of ceramide in the cell, thereby decreasing the amount of mSREBP in the cell.

This invention also provides a method for decreasing cholesterol synthesis in a cell characterized by an elevated level of mSREBP comprising contacting the cell with an agent that specifically inhibits de novo synthesis of ceramide in the cell, thereby decreasing cholesterol synthesis in the cell.

This invention also provides a method for decreasing fatty acid synthesis in a cell characterized by an elevated level of mSREBP comprising contacting the cell with an agent that specifically inhibits de novo synthesis of ceramide in the cell, thereby decreasing fatty acid synthesis in the cell.

This invention also provides a method for decreasing triglyceride synthesis in a cell characterized by an elevated level of mSREBP comprising contacting the cell with an agent that specifically inhibits de novo synthesis of ceramide in the cell, thereby decreasing triglyceride synthesis in the cell.

In all of the instant methods, the cell can be, for example, a human cell, a hepatocyte or an adipocyte. In another embodiment, the agent in the instant methods specifically inhibits the activity of an enzyme which catalyzes part of the de novo ceramide pathway. Such enzymes include, for example, serine-palmitoyl transferase and ceramide synthase. In another embodiment, the agent inhibits the expression of an enzyme which catalyzes part of the de novo ceramide pathway.

In the instant methods, agents include, for example, myriocin, cycloserine, Fumonisin B1, PPMP, compound D609, methylthiodihydroceramide, propanolol and resvaratrol. Agents also include, without limitation, antisense nucleic molecules directed against mRNA encoding an enzyme which (i) catalyzes part of the de novo ceramide pathway, (ii) nucleic acids encoding same, and (iii) antibodies and fragments thereof which bind to such enzymes.

This invention further provides a method for increasing the amount of mSREBP in a cell comprising contacting the cell with an agent that specifically increases de novo synthesis of ceramide in the cell, thereby increasing the amount of mSREBP in the cell.

This invention provides a method for treating a subject afflicted with a disorder characterized by an elevated level of mSREBP in the subject's cells comprising administering to the subject a therapeutically effective amount of an agent that specifically inhibits de novo synthesis of ceramide in the subject's cells, thereby treating the subject.

This invention also provides a method for treating a subject afflicted with a disorder characterized by increased ceramide synthesis in the subject's cells comprising administering to the subject a therapeutically effective amount of an agent that specifically inhibits de novo synthesis of ceramide in the subject's cells, thereby treating the subject.

This invention also provides a method for treating a subject afflicted with an elevated cholesterol level comprising administering to the subject a therapeutically effective amount of an agent that specifically inhibits de novo synthesis of ceramide in the subject's cells, thereby treating the subject.

This invention further provides a method for treating a subject afflicted with an elevated fatty acid level comprising administering to the subject a therapeutically effective amount of an agent that specifically inhibits de novo synthesis of ceramide in the subject's cells, thereby treating the subject.

This invention further provides a method for treating a subject afflicted with an elevated triglyceride level comprising administering to the subject a therapeutically effective amount of an agent that specifically inhibits de novo synthesis of ceramide in the subject's cells, thereby treating the subject.

This invention also provides a method for inhibiting in a subject the onset of a disorder characterized by an elevated level of mSREBP in the subject's cells comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by increased ceramide synthesis in the subject's cells comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by an elevated cholesterol level in the subject comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by an elevated fatty acid level in the subject comprising administering to the subject a prophylactically effective amount of an agent that specifically inhibits de novo synthesis of ceramide in the subject's cells, thereby inhibiting the onset of the disorder.

This invention also provides a method for inhibiting in a subject the onset of a disorder characterized by an elevated triglyceride level in the subject comprising administering to the subject a prophylactically effective amount of an agent that specifically inhibits de novo synthesis of ceramide in the subject's cells, thereby inhibiting the onset of the disorder.

In the preferred embodiment of the instant methods, the subject is a human. In one embodiment, the subject has a lipid disorder. Lipid disorders include, without limitation, hypercholesterolemia, hypertriglyceridemia, combined familial hyperlipidemia, obesity, type I diabetes, type II diabetes, alcoholism, metabolic syndrome, syndrome X, hypertension and cardiovascular disease.

In another embodiment, the disorder is selected from the group consisting of Hereditary Sensory Neuropathy, Niemann Pick Disease Type A (including heterozygous carrier of Niemann Pick Disease Type A) and Niemann Pick Disease Type B (including heterozygous carrier of Niemann Pick Disease Type B).

The agent used in the instant methods can be, for example, myriocin, cycloserine, Fumonisin B1, PPMP, compound D609, methylthiodihydroceramide, propanolol or resvaratrol.

Therapeutically and prophylactically effective amounts of an agent for humans can be determined from animal data using routine computational methods. In one embodiment, the therapeutically or prophylactically effective amount of an agent is an amount sufficient to give rise to a cellular concentration of between 10 nM and 1 mM. In another embodiment, the therapeutically or prophylactically effective amount of an agent is an amount sufficient to give rise to a cellular concentration of between 100 nM and 100 μM. In another embodiment, the therapeutically or prophylactically effective amount of an agent is an amount sufficient to give rise to a cellular concentration of between 1 μM and 50 μM.

In one embodiment, therapeutically or prophylactically effective amounts of agents used in the instant methods are amounts sufficient to give rise to cellular concentrations as follows: (a) myriocin, 0.1-10 μM; (b) cycloserine, 0.5-5 mM; (c) fumonisin B1, 0.1-40 μM; (d) PPMP, 0.5-50 μM; (e) compound D609, 10-80 μg/ml; (f) methylthiodihydroceramide, 10-50 μM; (g) propanolol, 100-500 mM; and (h) resvaratrol, 150-600 mM.

This invention provides a method for increasing the amount of mSREBP in the cells of a non-human subject comprising administering to the subject an effective amount of an agent that specifically increases de novo synthesis of ceramide in the subject's cells, thereby increasing the amount of mSREBP in the subject's cells.

This invention also provides a first article of manufacture comprising a packaging material having therein an agent that specifically inhibits de novo synthesis of ceramide in a cell, and a label indicating a use for the agent in treating or inhibiting the onset of a disorder in a subject, which disorder is characterized by an elevated level of mSREBP in the subject's cells.

This invention further provides a second article of manufacture comprising a packaging material having therein an agent that specifically inhibits de novo synthesis of ceramide in a cell, and a label indicating a use for treating or inhibiting the onset of an elevated cholesterol level in a subject.

This invention also provides an article of manufacture comprising a packaging material having therein an agent that specifically inhibits de novo synthesis of ceramide in a cell, and a label indicating a use for treating or inhibiting the onset of an elevated fatty acid level in a subject.

This invention also provides an article of manufacture comprising a packaging material having therein an agent that specifically inhibits de novo synthesis of ceramide in a cell, and a label indicating a use for treating or inhibiting the onset of an elevated triglyceride level in a subject. Preferably, the instant articles of manufacture further comprise a pharmaceutically acceptable carrier.

This invention provides a method for determining whether an agent decreases de novo synthesis of ceramide in a cell, which method comprises the steps of (a) contacting the cell with the agent under suitable conditions; (b) determining the amount of de novo synthesis of ceramide in the cell after a suitable period of time; and (c) comparing the amount of de novo synthesis of ceramide determined in step (b) with the amount of de novo synthesis of ceramide in a cell in the absence of the agent, a lower amount of de novo synthesis of ceramide in the cell contacted with the agent indicating that the agent decreases the amount of de novo synthesis of ceramide in the cell.

Finally, this invention provides a method for determining whether an agent increases de novo synthesis of ceramide in a cell, which method comprises the steps of (a) contacting the cell with the agent under suitable conditions; (b) determining the amount of de novo synthesis of ceramide in the cell after a suitable period of time; and (c) comparing the amount of de novo synthesis of ceramide determined in step (b) with the amount of de novo synthesis of ceramide in a cell in the absence of the agent, a greater amount of de novo synthesis of ceramide in the cell contacted with the agent indicating that the agent increases the amount of de novo synthesis of ceramide in the cell. In these methods, suitable periods of time after which ceramide de novo synthesis is measured are exemplified in the Experimental Details section.

This invention will be better understood from the Experimental Details that follow. However, one skilled in the art will readily appreciate that the specific methods and results discussed are merely illustrative of the invention as described more fully in the claims which follow thereafter.

Experimental Details

The effect of decreasing cellular synthesis of ceramide on SREBP levels and SRE-mediated gene transcription was investigated. Increased cellular ceramide decreases mSREBP protein levels and SRE-mediated gene transcription (10). In keeping with the inhibitory effect of high levels of ceramide on SRE-mediated gene transcription, it was anticipated that inhibition of ceramide de novo synthesis should increase mSREBP levels and SRE-mediated gene transcription. Contrary to this hypothesis, it was found that inhibition of ceramide de novo synthesis decreases SRE-mediated gene transcription. Thus, the effect of ceramide on its own synthesis was investigated, and it was shown that exogenous or endogenous ceramide exerts a negative feed-back mechanism on its own synthesis. By the same token, increasing ceramide de novo synthesis correlates with increased mSREBP levels and SRE-mediated gene transcription. The role of ceramide de novo synthesis in SRE-mediated gene transcription is supported by experiments in cells that lack ceramide de novo synthesis (LY-B cells) due to a mutation in the LCB1 subunit of serine-palmiltoyl transferase (21, 22). LY-B cells fail to increase SRE-mediated gene transcription when they are cholesterol depleted. Since ceramide increases levels for pSREBP but decreases levels for mSREBP, it is suggested that ceramide blocks the maturation cascade of SREBP. The data presented here provide evidence that ceramide de novo synthesis is an important regulatory factor in the maturation cascade of SREBP.

### Part I

Experimental Procedures and Design

Materials: ³H-serine (555 Gbq-1.48 TBq; 0.1 mCi/mmol) and ³H-sphingosine (555 Gbq-1.11 TBq; 0.1 mCi/ml) were purchased from Perkin Elmer (Boston, Mass.). Chinese hamster ovary (CHO) cells were obtained from American Type Culture Collection (Rockville, Md.). LY-B cells (CHO cells with a mutation in the lcb1 subunit of serine-palmiltoyl transferase) were obtained from National Institutes of Infectious Diseases, Tokyo, Japan (21). Ethanol, fatty acid free bovine serum (BSA), cholesterol, 25-hydroxycholesterol (25-OH cholesterol), fumonisin B1 were obtained from Sigma, (St. Louis, Mich.). D-MAPP (1S,2R)-D-erythro-2-(N-Myristoylamino)-1-phenyl-1-propanol), C6-ceramide (D-erythro hexanoylsphingosine), C8-ceramide (D-erythro N-octanoylsphingosine), C6-dihydroceramide (D-erythro N-hexanoyldihydrosphingosine), NB-DNJ, (N-Butyldeoxynojirimycin-HCL), PPMP (DL-threo-1-Phenyl-2-palmitoylamino-3-morpholino-1-propanol HCL), and DMS (N,N-Dimethylsphingosine) were obtained from Biomol Research Laboratories, Inc. (Plymouth Meeting, Pa.). All cell culture reagents and neomycin (G418) were obtained from Life Technologies, Inc. (Grand Island, N.Y.). All organic solvents were purchased from Fisher Scientific Co. (Springfield, N.J.).

Plasmids: The pSyn-SRE plasmid contains a generic TATA-box and three SRE elements (−326 to −225 bp) of the hamster HMG-CoA synthase promoter fused into the luciferase pGL2 Basic vector (Promega, Madison, Wis.) and has been described before (4, 23). The pWLNeo plasmid was obtained from Stratagene Inc. (La Jolla, Calif.).

Cell culture and stable transfections: Cells were grown in F12-nutrient mixture medium containing 10% fetal bovine serum (FBS), 1% glutamine (v/v), 1% penicillin/streptomycin (v/v), and 10% fetal bovine serum (v/v) at 37° C. in humidified CO₂ (5%). To obtain stable transfectants, cells were plated in 12-well plates at 50% confluency and transfected for 5 h in the presence of serum-free Dulbecco's modified Eagle's medium (DMEM) with pSyn SRE (1 μg/well) and pWLNeo (0.25 μg/well) using Lipofectamin (1.5 μl/well). Cells were then incubated for 2 days in growth medium. On day three, neomycin-containing medium (400 μg/ml) was added. Selection for neomycin resistant colonies was continued for three weeks. Pooled clones were analyzed for luciferase expression. Experiments were performed with pooled clones as well as with cells derived from a single clone. Cells were grown in the presence of 400 μg/ml neomycin. For experimental use, cells were plated in the absence of neomycin at least 24 h ahead in regular growth medium.

Enzyme assays: Cells to be analyzed for luciferase activity were lyzed in lysis buffer A containing 0.1% Triton X-100, 50 mM Hepes, 10 mM MgSO4, pH 7.7. Cells were scraped, collected, vortexed and briefly centrifuged to pellet cell debris. An aliquot was used to measure luciferase activities in a luminometer (Berthold LB 9501, Wallac Inc., Gaithersburg, Md.) with a luciferin reagent from Promega (Madison, Wis.). Luciferase activity in relative light units (RLU) was divided by protein content (mg/ml) for each extract.

Protein determination: The amount of cellular protein was determined by the Biorad method and BSA was used as a standard.

Measurement of cell survival by 3-(4,5-Dimethylthiazol-2yl)-2-5 diphenyltetrazolium bromide (MTT): All conditions not previously (10) evaluated for cell survival were determined by using the MTT assay (24). Cells (5×10³ cells/well) were plated into 96-well plates containing growth medium. The next day, cells were incubated for 8 h with sphingosine, D-erythro-dihydrosphingosine, DMS, PPMP, cycloserine, fumonisin B1 in the presence of 200 μl 1% fatty acid-free BSA dissolved in serum-free Ham's F12 medium at 37° C. in 5% CO₂. They were then treated with 20 μl of MTT for 4 h. Medium was discarded and cells were incubated for 5 min with 150 μl of DMSO. Then plates were read in a microplate reader (Labsystem Multiskan, Fisher Scientific, Morris Plains, N.J.) at 540 nm. Cell viability was also assessed by the trypan blue exclusion method. Cells were incubated with 0.2% trypan blue and cells that exclude trypan blue were counted using a hematocytometer determining the percentage of viable cells.

Ceramide de novo synthesis: Cells were plated in 6-well plates and incubated in the experimental conditions. During the last 1.5 h, ³H-serine (1 μl/ml) was added to allow incorporation into ceramide (25, 26). After the incubation, cells were washed two times with PBS, 0.2% BSA and two times with PBS alone. Then cells were lyzed in 400 μl lysis buffer B (250 mM Tris-Cl), scraped and transferred to glass tubes. An aliquot was used for luciferase and protein determination. Then, 1 ml of ice-cold methanol, 2 ml of chloroform and 0.5 ml of 0.1 N HCl was added, vortexed and spun at 800 g for 10 min. The upper phase was discarded and the organic phase was washed with 3×2 ml 0.001 N HCl. Lipids were then dried under N₂. Alkaline hydrolysis was performed by incubation in 2 ml of 0.1 N KOH in methanol at 37° C. for 1 h. Lipids were then reextracted by adding 2 ml of chloroform and 1.2 ml of balanced salt solution (135 mM NaCl, 4.5 mM KCl, 1.5 mM CaCl₂, 0.5 mM MgCl₂, 5.6 mM Glucose, 10 mM Hepes, pH 7.2)/EDTA 100 mM (1.08 ml/0.12 ml). After vortexing and centrifugation at 800×g for 5 min, the lower phase was dried under N₂ (27, 28). The extracted lipids were then dissolved in 50 μl chloroform/methanol (1:1) spotted on TLC plates (Merck Silicagel 60, Darmstadt, Germany) and chromatographed with chloroform-methanol-0.22% aqueous CaCl₂ (60:35:8 v/v) (29). Ceramide and sphingomyelin (dissolved at 1 μg/μl) were run as standards. The lipids were identified according to their Rf values after visualization in an iodine vapor tank. The TLC plate was cut at the corresponding lipid spots, mixed with scintillation fluid (Ultima Gold, Packard Instrument Company, CT) and analyzed in a scintillation counter (Perkin Elmer Wallac, Gaithersburg, Md.). Results were expressed in dpm/mg protein as a percentage of total counts. Absolute cpm values range between 400 and 800 cpm. Incorporation of ³H from ³H-serine over 1.5 h into fatty acids, cholesterol, triglycerides or cholesterol ester was less than 5% and not significant. Determination of sphingomyelin levels were carried out using standard TLC methods (30). In brief, lipids were extracted as above. Lipid extracts were run on TLC. The spots corresponding to sphingomyelin were eluted and levels of phosphorus were determined using standard methods (31).

Western blot analysis: Cells were plated on day one in regular growth medium. On day two, cells were incubated in control media (1% BSA, fatty acid-free) or with the respective conditions. Two hours before harvesting, all cells received 25 μg/ml N-acetyl-leucyl-leucyl-norleucinal (ALLN) to inhibit proteolysis of SREBP by the proteasome. After 8 hours, cells were scraped and pelleted at 1000×g. The pellet was resuspended in lysis buffer C (10 mM Tris-Cl, 100 mM NaCl, 1% SDS, pH 7.6) containing protease inhibitors COMPLETE™ (Roche Pharmaceuticals, Nutley, N.J.). An aliquot of each sample (30 μg of protein) was subjected to electrophoresis on a denaturing 7.5% sodium dodecyl sulfate polyacrylamide gel electrophoresis (SDS-PAGE). The monoclonal antibodies against SREBP-1 or SREBP-2 (BD Biosciences, San Jose, USA) and actin (Sigma, St. Louis, Mich.) and the peroxidase labeled anti-mouse IgG (Amersham NIF 824) were used for Western blot analysis according to the manufacturer's instructions. Detection was performed with the ECL method (Amersham, Arlington Heights, Ill.). Protein mobilities were compared to prestained broad-range molecular weight standards (Biorad, Hercules, Calif.). Densitometric quantification was carried out using Scion Image beta 4.02 software (www.scioncorp.com).

Northern blots: CHO cells were plated on day 1 at 80% confluency and treated with the respective conditions for 8 h. Total RNA was isolated by Trizol reagent (Invitrogen, Grand Island, N.Y.) as described by the manufacturer. RNA concentration was calculated from optical density at 260 nm. 30 μg of total RNA were separated by 1.2% denaturing agarose/formaldehyde electrophoresis and transferred by capillary transfer to Duralon UV-membranes (Stratagene, La Jolla, Calif.). The cDNA probe for northern hybridization of HMG-CoA synthase was obtained by RT-PCR from human THP-1 macrophages mRNA using previously described primers (4). The blot was hybridized in Quick-Hyb (Stratagene, La Jolla, Calif.) for 1 h with cDNA probes corresponding to HMG-CoA synthase and glyceraldehyde-3-phosphate dehydrogenase corresponding to bases 247-882 as a loading control. Probes were labeled by random priming (Stratagene PRIME-IT® Random priming labeling kit) using 50 μCi of α³²CTP (3000 Ci/mmol) and 50 ng of DNA fragment.

Data Analysis: Statistical significance was calculated by paired t-tests. Unless otherwise indicated, results are given as mean±S.D. All experiments were repeated on different days at least 3 times and each time in triplicate.

Results

Applicants have previously shown that unsaturated fatty acid-mediated decrease of SRE-mediated gene transcription is linked to sphingolipid metabolism. Applicants also showed that increasing levels of cellular ceramide, through addition of cell-permeable ceramide and dihydroceramide analogues or by inhibition of ceramidase, decreases SRE-mediated gene transcription and cellular levels of mSREBP (10). Applicants now investigate potential mechanisms for this effect.

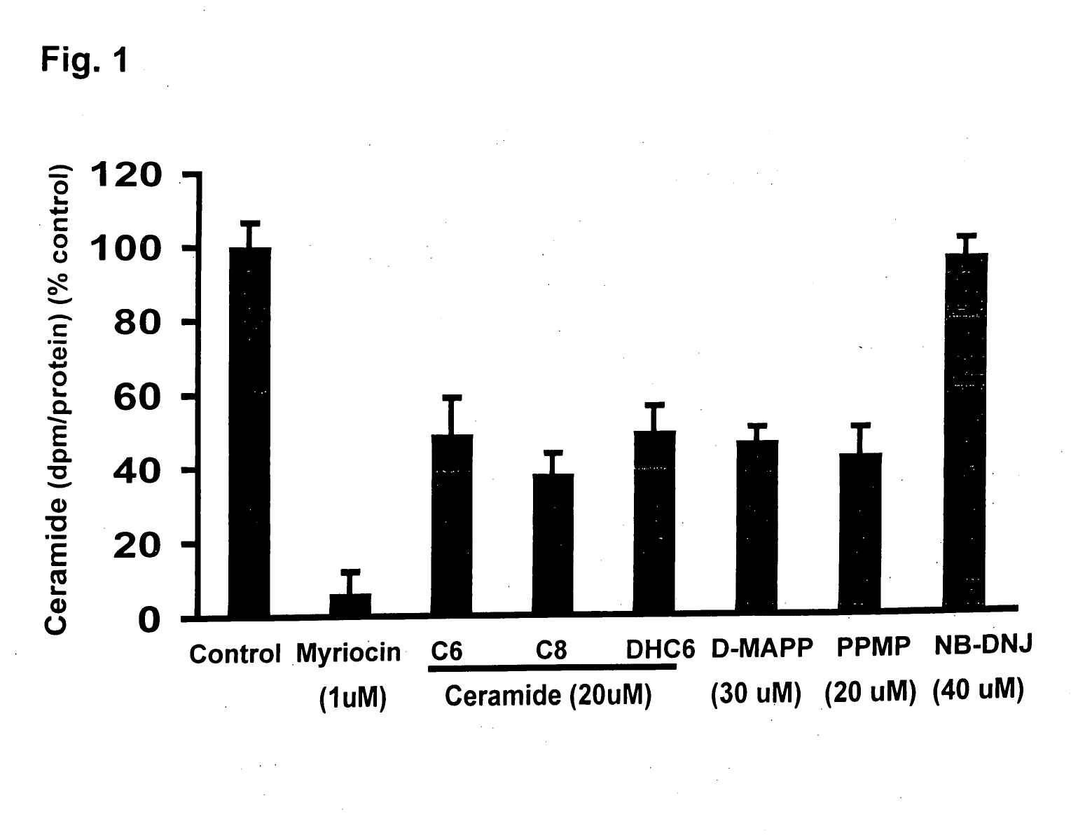

Increased exogenous and endogenous ceramide decreases ceramide de novo synthesis—Applicants first investigated whether increasing cellular ceramide levels decrease ceramide de novo synthesis. Cells were incubated for 8 h in the presence of C6- or C8-ceramide (20 μM), DH-C6-ceramide (20 μM), D-MAPP (20 μM) an inhibitor of alkaline ceramidases or PPMP (20 μM), an inhibitor of glucosylceramide synthesis (32). As a negative control, cells were incubated with NB-DNJ (40 μM), an inhibitor of glucosylceramide synthesis that does not increase ceramide levels (33). For the last 1.5 h of incubation time, ³H-serine was added as a label to determine ceramide de novo synthesis. All conditions, except incubation with NB-DNJ, significantly (p<0.05) decreased ceramide de novo synthesis measured by incorporation of ³H-serine into ceramide (FIG. 1). None of the conditions used significantly affected cell survival, measured as outlined under ‘Experimental Details.’

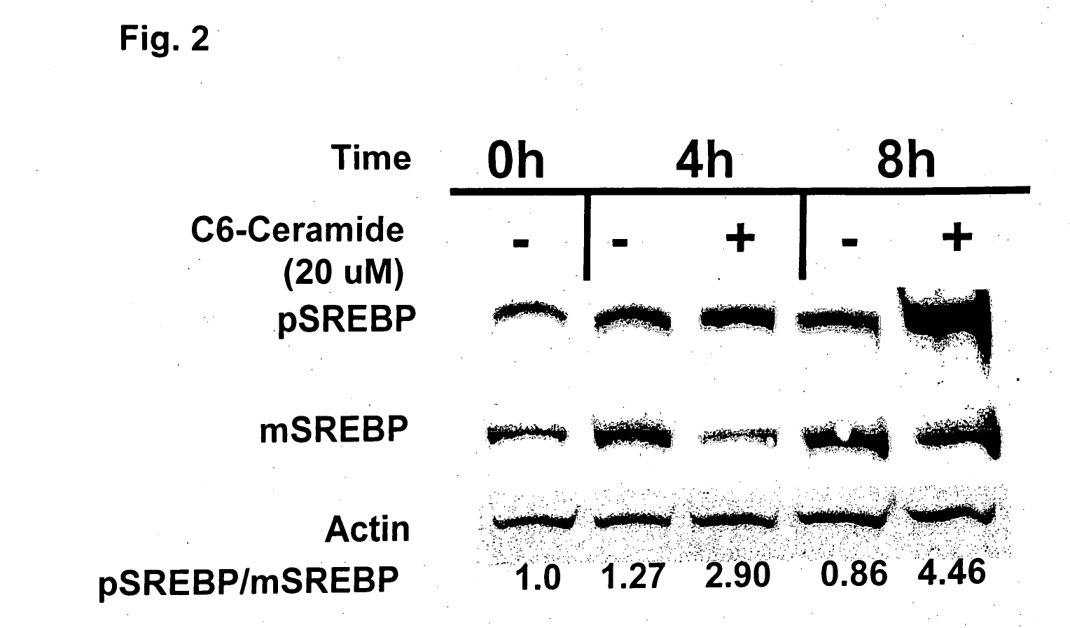

Ceramide increases levels of precursor SREBP and decreases levels of mature SREBP—To investigate the effect of decreased ceramide de novo synthesis on cellular levels of SREBP, western blot analysis was carried out. Incubation of CHO cells over 4 h and 8 h with C6-ceramide (20 μM) increased cellular levels of pSREBP compared to controls at 4 h and even more at 8 h (FIG. 2). At the same time, levels of mSREBP decreased in the presence of C6 ceramide at 4 h and at 8 h compared to controls and compared to pSREBP of the same cell extract. To assure equal loading of the gel, the membrane was also probed for actin, which was not affected by addition of C6-ceramide. Applicants have previously shown that ceramide inhibits the generation of the mature form of SREBP. These data suggest that ceramide analogues inhibit the processing of pSREBP to mSREBP and induce an accumulation of pSREBP.

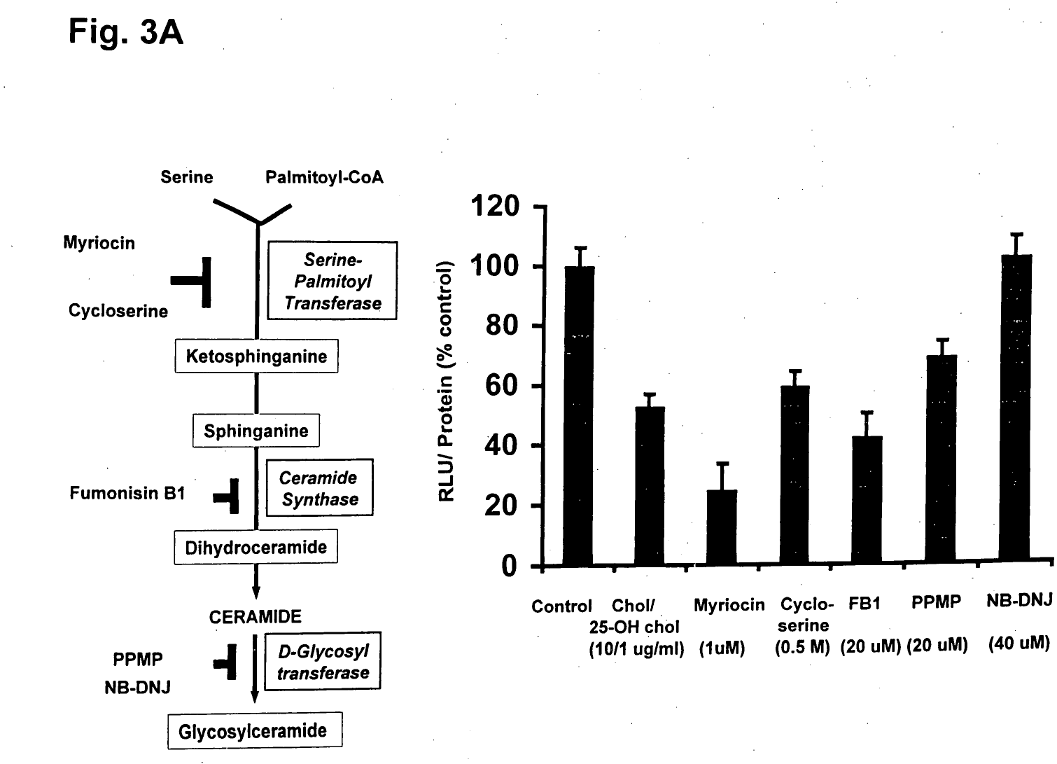

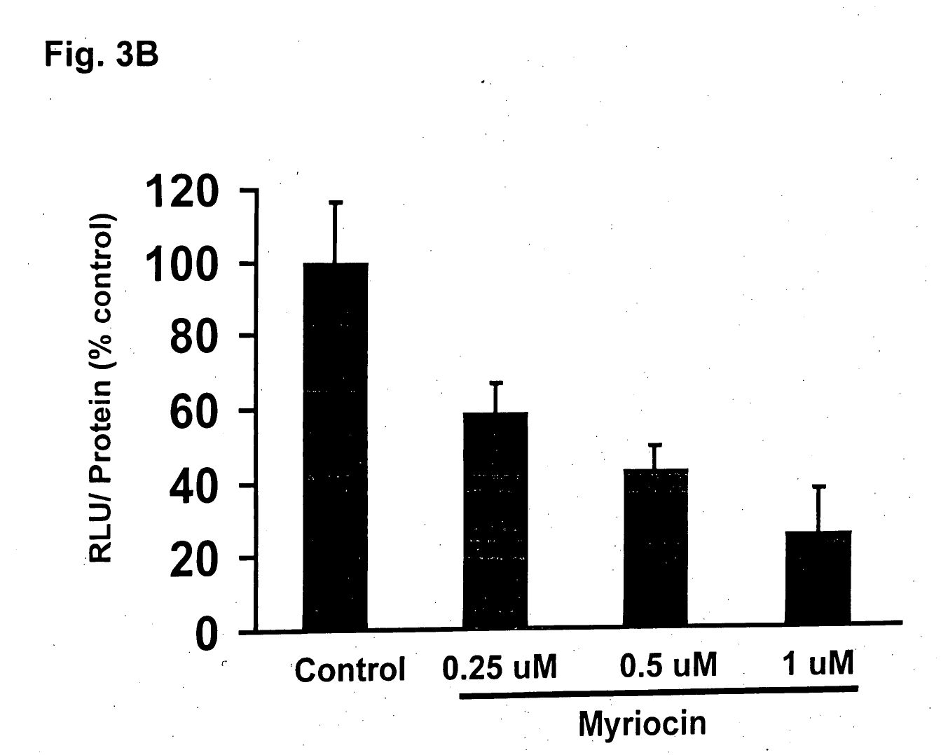

Inhibition of ceramide de novo synthesis decreases SRE-mediated gene transcription—Applicants next investigated the effect of decreased de novo ceramide synthesis on SRE-mediated gene transcription (FIG. 3). Chinese hamster ovary cells (CHO) that are stable transfectants for a SRE-regulated promoter linked to the luciferase reporter gene were incubated for 8 hours with myriocin (1 μM), a specific inhibitor of serine-palmitoyl transferase (34), cycloserine (500 mM), another inhibitor of serine-palmitoyl transferase (35, 36), or fumonisin B1 (10 μM), an inhibitor of ceramide synthase (37). All three inhibitors significantly reduced SRE-mediated gene transcription at 8 h (FIG. 3A). Cells were also incubated with PPMP (20 μM), a glucosyltransferase inhibitor which increases intracellular ceramide levels and decreases ceramide de novo synthesis as measured by incorporation of ³H-serine (FIG. 1). As a negative control, cells were incubated with 40 μM NB-DNJ, an inhibitor of glucosylceramide synthase that does not increase ceramide (33). The effects of myriocin were dose-dependent (FIG. 3B) and reversible within 8 h and did not decrease the expression of another control β-gal reporter gene (data not shown). Addition of ceramide under the experimental conditions depicted in FIG. 3 did not increase SRE-mediated gene transcription (data not shown).

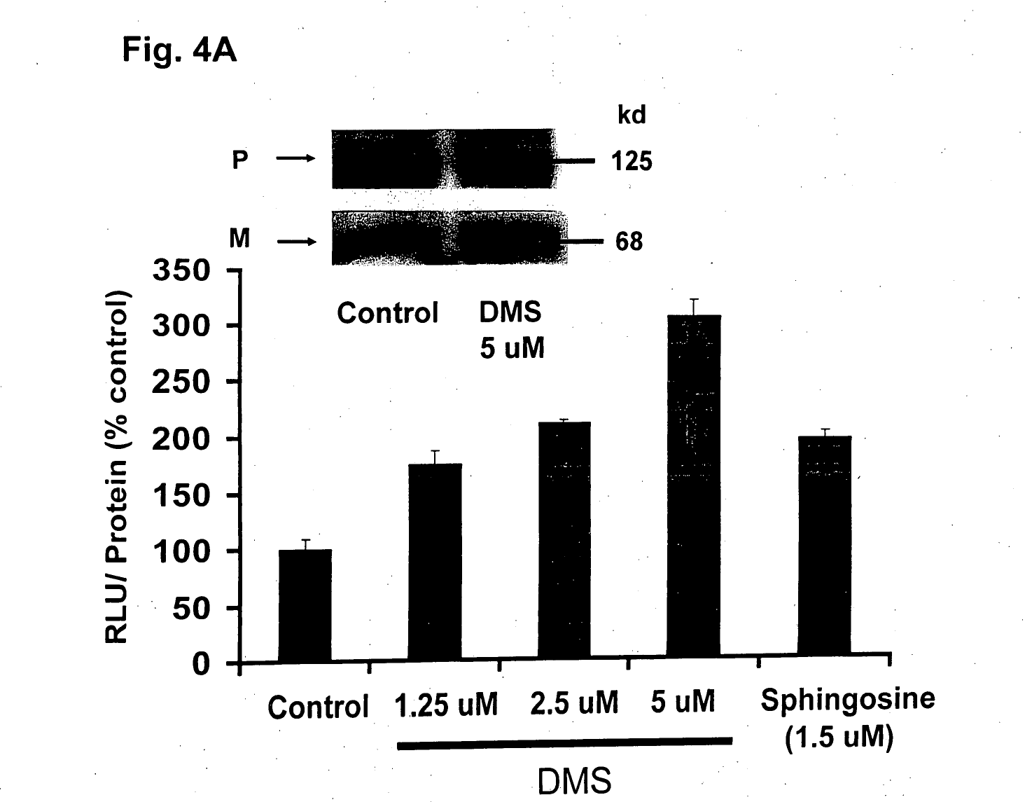

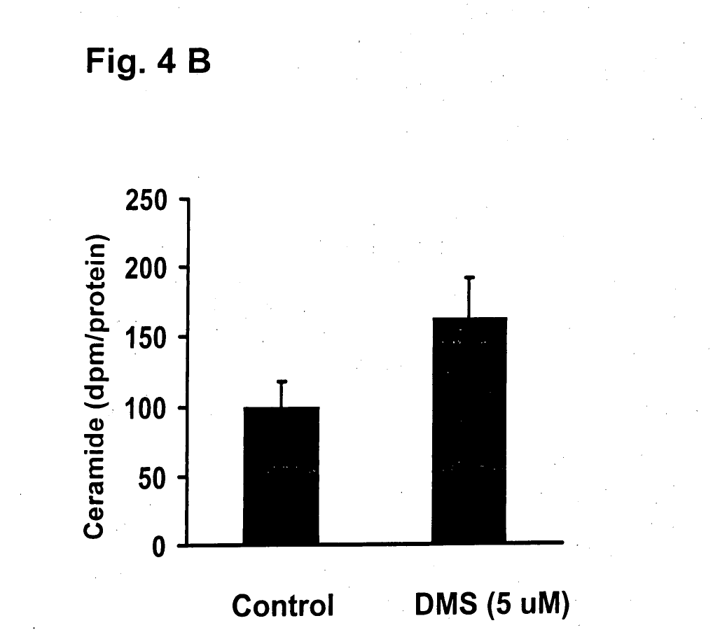

Increased ceramide de novo synthesis increases SRE-mediated gene transcription—Sphingosine is a precursor of ceramide and increases ceramide de novo synthesis (38-40). Endogenous sphingosine levels are also increased by DMS, an inhibitor of sphingosine-1-phosphate kinase (41, 42). Cellular sphingosine levels were increased by addition of sphingosine (1.5 μM) within 8 h or by incubation with DMS (1.5-5 μM) for up to 8 h. DMS dose-dependently increases SRE-mediated gene transcription (FIG. 4A). Levels of mSREBP also increased (FIG. 4A inset). Sphingosine also increases SRE-mediated gene transcription up to two-fold (FIG. 4A). DMS dose-dependently increases incorporation of ³H-sphingosine label into ceramide by 70% within 5 h (FIG. 4B). The data demonstrate that increased ceramide synthesis correlates with an increase in SRE-mediated gene transcription and mSREBP levels.

LY-B cells that do not synthesize de novo ceramide fail to increase SRE-mediated gene transcription with sterol depletion—Next, applicants examined the role of ongoing ceramide de novo synthesis in the processing of SREBP in a cell line that does not produce ceramide by the de novo pathway (LY-B cells) (21). LY-B cells have a mutation in the LCB1 subunit of serine-palmitoyl transferase that results in a complete lack of serine-palmiltoyl transferase activity with subsequent inability to de novo synthesize any sphingolipid species. For normal growth, LY-B cells depend on the recycling pathway of sphingolipids. Importantly, LY-B cells have normal cellular ceramide and free cholesterol levels and cellular sphingomyelin levels are decreased (21, 22).

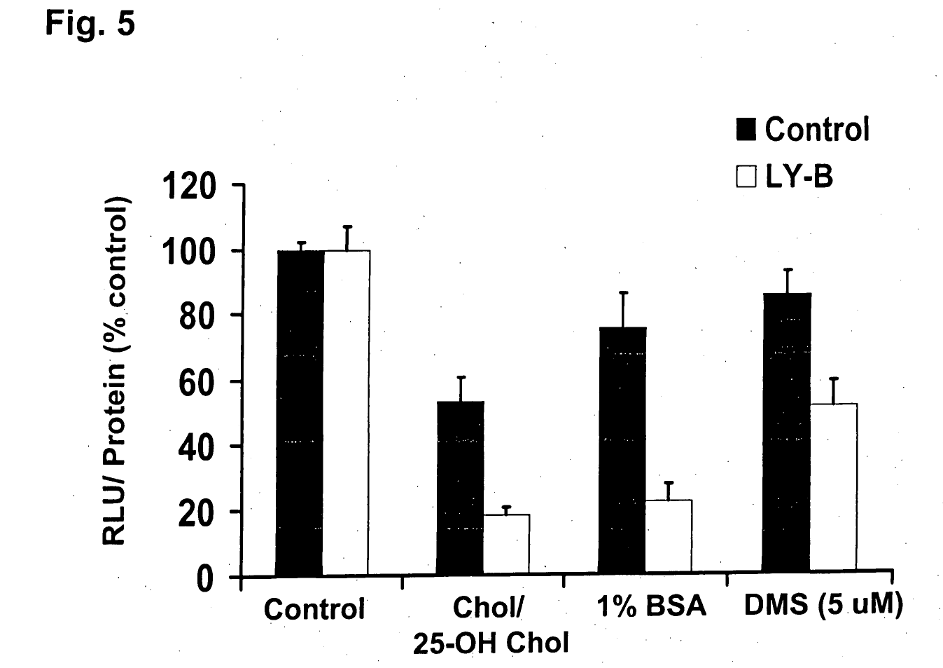

SRE-mediated gene transcription was first suppressed by incubation for 16 h in the presence of cholesterol (10 μg/ml) and 25-OH cholesterol (1 μg/ml). Then, cells were switched for 6 h to medium containing 1% BSA. Control cells increased SRE-mediated gene transcription but LY-B cells failed to do so (FIG. 5). Control experiments were carried out to demonstrate that LY-B cells are able to increase SRE-mediated gene transcription once a precursor for ceramide synthesis is supplied. Again, LY-B and control cells were incubated in the presence of cholesterol and 25-OH cholesterol for 16 h, then medium was switched to 1% BSA containing 5 μM DMS. Within 6 h, cells significantly increased SRE-mediated gene transcription comparable to control cells, indicating that when the block in de novo ceramide synthesis is bypassed by DMS that SREBP cleavage returns towards normal. Addition of DMS together with fumonisin B1 or PPMP does not lead to an increase in SRE-mediated gene transcription in LYB cells (data not shown). In preliminary data, incubation of LYB-cells with ceramide did not decrease SRE-mediated gene transcription, emphasizing the importance of functional serine-palmitoyl transferase in the regulation of SREBP maturation.

Thus, several lines of evidence demonstrate that ceramide de novo synthesis correlates with SRE-mediated gene transcription. SRE-mediated gene transcription is decreased when ceramide de novo synthesis is decreased secondary to inhibition of serine-palmitoyl transferase, ceramide synthase (FIG. 3), or ceramide mediated feed-back inhibition (10). In contrast, exogenous or endogenous sphingosine, two conditions that increase ceramide de novo synthesis, correlate with an increase in SRE-mediated gene transcription (FIG. 4). Data obtained with inhibitors and stimulators of ceramide de novo synthesis are confirmed in LY-B cells. LY-B cells, which cannot synthesize ceramide de novo, fail to increase SRE-mediated gene transcription in sterol depletion and absence of an exogenous source of sphingolipids (FIG. 5).

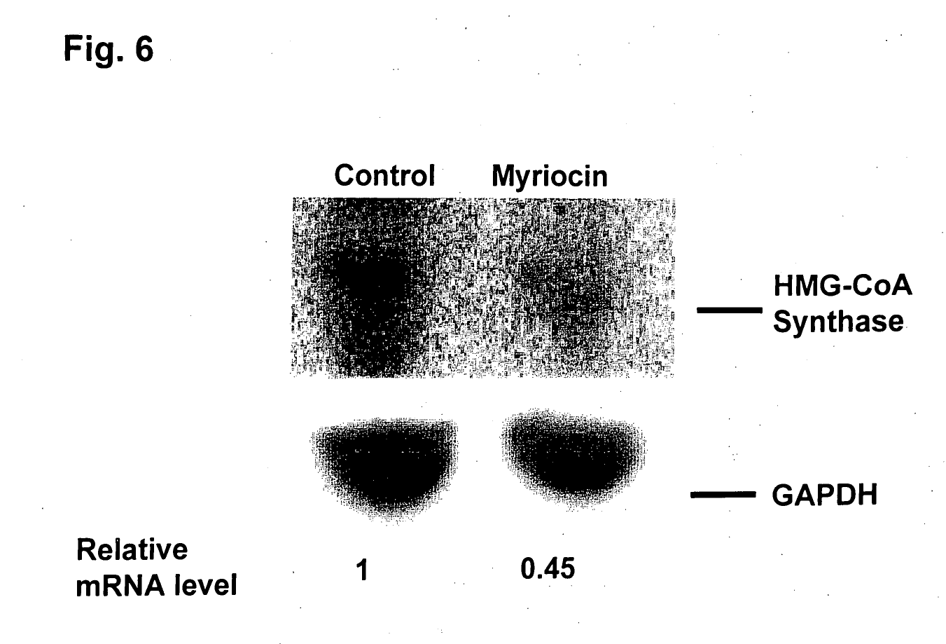

Inhibition of ceramide de novo synthesis decreases levels of HMG-CoA synthase mRNA—Applicants next examined if the results obtained with SRE-reporter gene assays reflect changes in the regulation of HMG-CoA synthase mRNA, a gene known to be sensitively regulated by the sterol regulatory element (43). Applicants have previously shown that ceramide and D-MAPP decrease mRNA levels of HMG-CoA synthase (10). Incubation with myriocin for 16 h equally decreases HMG-CoA synthase mRNA levels to half (FIG. 6). Therefore, changes in mSREBP and SRE-mediated gene-transcription are reflected in expression of genes dependent on pathways that regulate ceramide de novo synthesis.

Discussion

Applicants investigated potential mechanisms by which ceramide decreases levels of transcriptionally active mature SREBP and SRE-mediated gene transcription. Previously, applicants have shown that increasing cellular ceramide levels either by addition of ceramide analogues, increasing sphingomyelin hydrolysis or inhibition of intracellular ceramide metabolism decreases SRE-mediated gene transcription (10). The data suggested a cholesterol-independent regulatory mechanism of SREBP. Here, applicants demonstrate that ongoing ceramide de novo synthesis is required in the post-transcriptional regulation of SREBP and that ceramide-mediated regulation of SREBP and SRE-mediated gene transcription is linked to inhibition of ceramide de novo synthesis.

Increased cellular levels of ceramide decrease SRE-mediated gene transcription (10). Therefore, applicants' initial hypothesis was that through decreasing cell ceramide synthesis SRE-mediated gene transcription should be increased. Contrary to this initial hypothesis, inhibition of ceramide de novo synthesis correlated with a decrease in SRE-mediated gene transcription and decreased levels of mSREBP (FIG. 3). Of note, myriocin, a very specific and potent inhibitor of serine-palmitoyl transferase, inhibited SRE-mediated gene transcription more than cholesterol and 25-OH cholesterol (FIG. 3). Thus, increasing cellular ceramide levels (10) or as applicants describe now decreasing ceramide synthesis both decrease SRE-mediated gene transcription. Alternatively, increasing de novo synthesis of ceramide is associated with increases in SRE-mediated gene transcription.

Short chain ceramides, dihydroceramides and dihydroceramide analogues all inhibit de novo sphingolipid synthesis (44, 45). Therefore, applicants questioned whether ceramide also inhibits its own de novo synthesis and whether the lack of ceramide de novo synthesis regulates SRE-mediated gene transcription. Applicants demonstrate that exogenous short-chain ceramides C6 and C8-ceramide, dihydroceramide, DMAPP (an inhibitor of ceramidase that increases cellular ceramide levels (46)) or PPMP, an inhibitor of glucosylceramide synthase all decrease ceramide de novo synthesis (FIG. 1). Importantly, NB-DNJ, a glucosylceramide synthase inhibitor that does not increase endogenous ceramide levels, does not affect incorporation ceramide de novo synthesis (FIG. 1) or SRE-mediated gene transcription (FIG. 3).

The regulatory role of ceramide de novo synthesis in the post-transcriptional processing of SREBP is further supported by several experimental approaches. First, when cellular sphingosine levels are increased exogenously or endogenously, SRE-mediated gene transcription increases within 8 h (FIG. 4). Secondly, cells that cannot synthesize sphingolipids (LY-B) (21, 22) fail to increase SRE-mediated gene transcription in sterol depletion but recover SRE-mediated gene transcription when DMS, which increases sphingosine, a direct precursor for ceramide de novo synthesis, is present in the incubation medium (FIG. 5).

What is the role of cellular sphingomyelin levels on SRE-mediated gene transcription? Myriocin inhibits serine-palmitoyl transferase but does not change cellular sphingomyelin levels within 24 h (data not shown). LY-B cells have decreased sphingomyelin levels. Yet, SRE-mediated gene transcription is decreased in LY-B cells as well as in myriocin treated cells. The data also localize the required enzymatic step that mediates SRE-mediated gene transcription to the synthesis of ceramide and suggest that earlier metabolic steps (i.e., synthesis of sphinganine or dihydroceramide) are not necessary in the processing of SREBP because DMS rescues SRE-mediated gene transcription in LY-B cells (FIG. 5). Taken together, the data suggest that ‘ongoing’ ceramide synthesis, a mechanism described by work in Riezman's group in relation to protein sorting and intracellular trafficking of GPI-anchored proteins in yeast, is required for SRE-mediated gene transcription (18-20, 47).

The data indicate that addition of exogenous ceramide increases levels of pSREBP and decreases levels of mSREBP (FIG. 2). This suggests that the processing of pSREBP to mSREBP is inhibited. Potentially, this could occur at multiple cellular sites, such as the movement of pSREBP within the ER, the movement of pSREBP to the Golgi apparatus and the activity of site-1 and site-2 protease. Regulation of pSREBP and conversion to mSREBP is initiated by vesicular transport together with SCAP to the Golgi, where two specific proteases cleave pSREBP and release the transcriptionally active mSREBP (2). Ceramide de novo synthesis has been shown to be obligatory in the ER to Golgi trafficking of GPI-anchored proteins in yeast (19, 20, 47). Of relevance, increased levels of ceramides inhibit the formation of coated vesicles in CHO cells (15), glycoprotein traffic through the secretory pathway (16) and decrease endocytosis in mammalian cells (17). Therefore, applicants hypothesize that de novo ceramide synthesis is important for the trafficking of pSREBP from ER to Golgi and its concomitant cleavage to mSREBP. It is possible that ceramide or changes in ceramide synthesis may lead to increase in pSREBP synthesis as it has been described for SREBP-2 in hamsters treated with mevinolin and colestipol (48). Of note, in this model there was also an increase in mSREBP-2. This possibility has not been ruled out and, due to the absolute decrease in mature SREBP resulting in decreased SREBP gene transcription (10), could be of lesser physiological importance.

Sphingolipids as well as cholesterol and sterols are known to modulate the physical properties of biological membranes. In applicants' experimental conditions, the effects of inhibitors of ceramide de novo synthesis were reversible and did not affect the expression of another control reporter gene. Because ceramide has been described to inhibit intracellular trafficking of glycoproteins (16) and to inhibit the generation of coated vesicle proteins in CHO cells (15), it is unlikely that the effect on SREBP trafficking is unique. Of note, sterols inhibit the protein trafficking across the endoplasmic reticulum membrane of proteins that are not closely related to cholesterol metabolism (49).

It has previously been reported that ceramide decreases mSREBP levels and SRE-mediated gene transcription (50), and this occurs even in the presence of inhibitors of intracellular cholesterol movement (10). Of interest, there is further evidence of a cholesterol-independent regulation of SREBP. Drosophila melanogaster SREBP levels are only regulated by palmitic acid but not by cholesterol or unsaturated fatty acids (9). Palmitic acid determines the rate of sphingosine and sphinganine synthesis (39), both important steps in ceramide formation. Hence ceramide synthesis may also contribute to SREBP regulation in Drosophila. In mammalian cells, SREBP formation and cleavage occur by a number of metabolic pathways—pathways that can be modified by diet or by therapeutic agents. These ‘regulators’ include cholesterol (51, 52), fatty acids (4, 5, 7) and as applicants show herein, modification of ceramide synthesis.

### Part II

The Effect of Different Inhibitors of Sphingolipid Synthesis on SRE-Mediated Gene Transcription

Background

In order to demonstrate experimentally the link between altered sphingolipid de novo synthesis and SREBP, two different disease models were investigated: Hereditary Sensory Neuropathy type 1 (HSN1) and Niemann Pick Disease Type A (NPA) and Type B (NPB).

In line with applicants' previous data that implicate the importance of de novo ceramide synthesis in the regulation of SREBP, several hypotheses were formulated: (1) HSN have increased SREBP activity (measured by SRE-mediated gene transcription); (2) HSN have increased cholesterol synthesis; (3) Pathology of HSN and possible other sensory neuropathies/neuropathies of small unmyelinated fibers, relates to/is secondary to cholesterol toxicity; and (4) Increased ceramide de novo synthesis affects SREBP and SRE-mediated gene transcription in Niemann Pick Type A cells.

Experimental Methods

Reduction of de novo sphingolipid synthesis: Incubation with methylthiodihydroceramide (10 μM) for 6 h reduces SRE-mediated gene transcription to 40%. Methylthiodihydroceramide was received from Gerhild van Echten-Deckert (1). Reduction of de novo sphingolipid biosynthesis by 1-methylthiodihydroceramide is due to its ability to deplete cells of newly formed free sphinganine. This compound does not induce an accumulation of precursors of sphingolipid de novo synthesis (as it is the case with fumonisin, which results in an accumulation of sphinganine). This experiment demonstrates that the inhibition of ceramide synthesis and not the accumulation of precursors (i.e., sphinganine) results in decreased SRE-mediated gene transcription.

Inhibition of sphingomyelin synthesis: The D609 compound is a xanthate and inhibitor of sphingomyelin synthase. It is a mixture of several isoforms (2). Experiments were carried out with five defined isomers of D609 received via Gemma Fabrias (Dept. of Biological Organic Chemistry, Barcelona, Spain) (Table 1). The goal was to test whether a specific isomer is more potent than another. A concentration curve of 10, 20 and 40 μg/ml was tested over 5 h.

TABLE 1

| | | Reduction of SRE- |
|---|---|---|
| | | mediated gene |
| Type | Form | transcription(40 μg/ml) |
| AG10 | endo—endo | 52% (±11%) |
| AG11 | exo-endo | 60% (±6%) |
| AG12 | endo-exo | 55% (±2%) |
| AG13 | exo—exo | 63% (±9%) |
| AG15 | | 65% (±6%) |

The results suggest that all forms moderately decrease SRE-mediated gene transcription. Also, the endo-endo and exo-endo forms reduce SRE-mediated gene transcription more than the endo-exo and exo-exo forms. The results indicate that possibly the step leading from ceramide to sphingomyelin (and the generation of a potential intermediate, such as diacylglycerol; see below) has an effect on SRE-mediated gene transcription. It is likely that inhibitors of sphingomyelin synthase would inhibit de novo synthesis of ceramide because they increase intracellular ceramide levels. Increased cellular ceramide levels have been shown to decrease ceramide de novo synthesis.

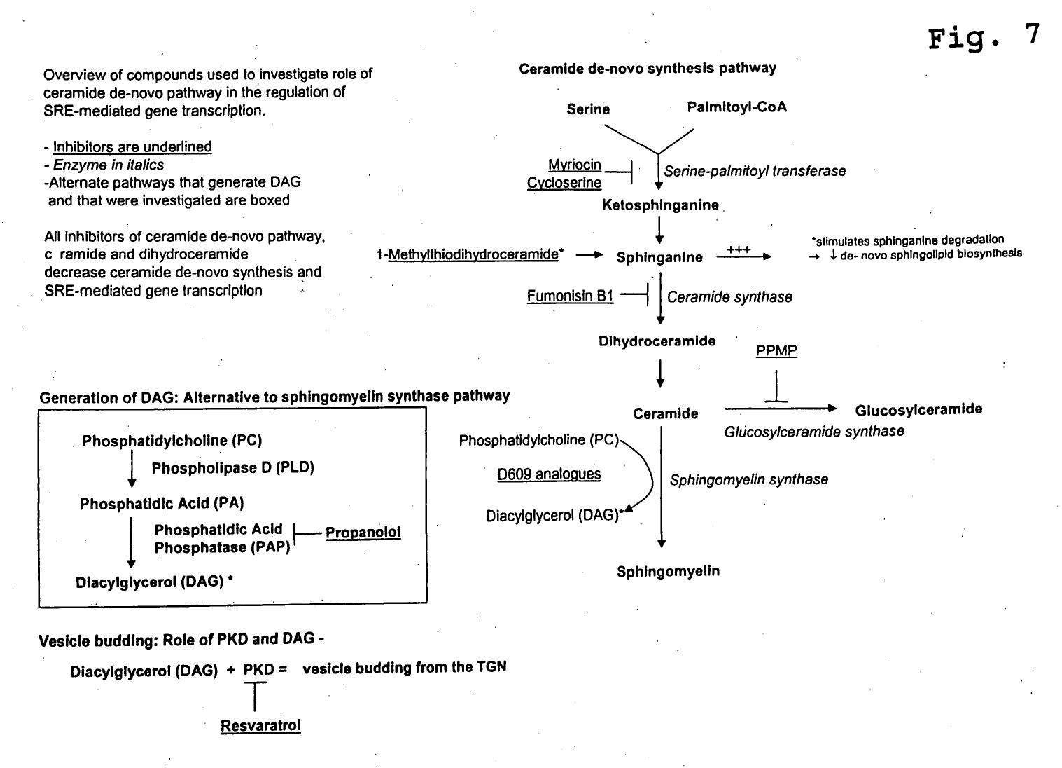

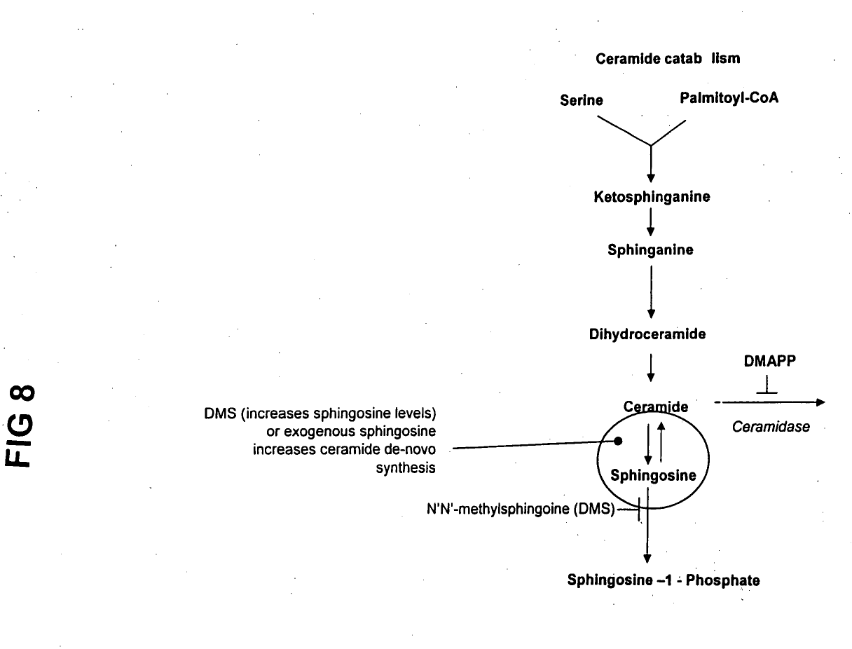

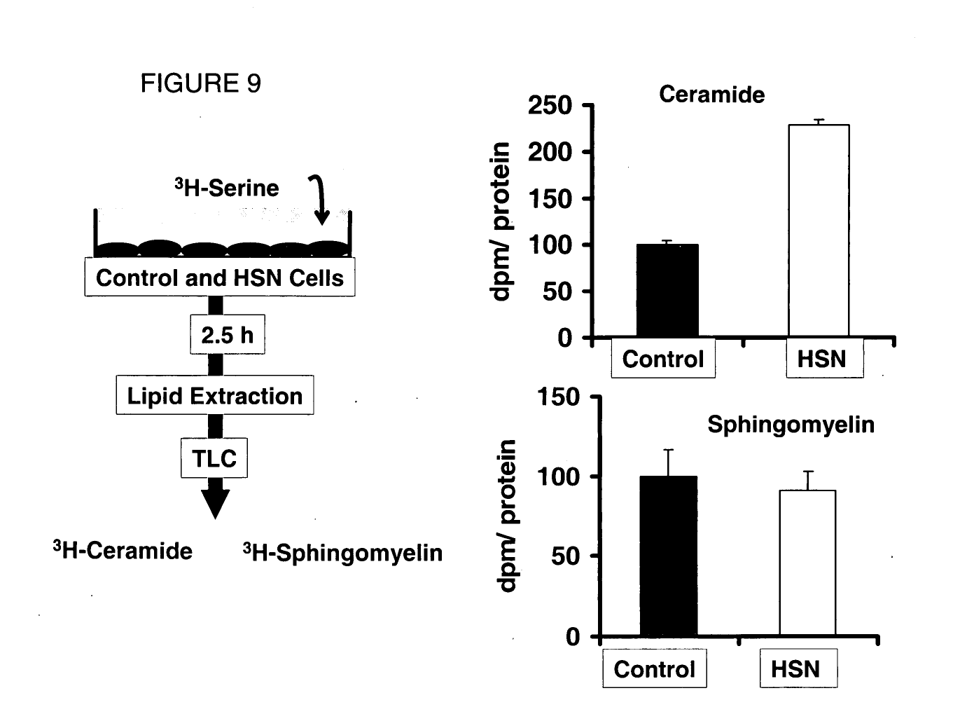

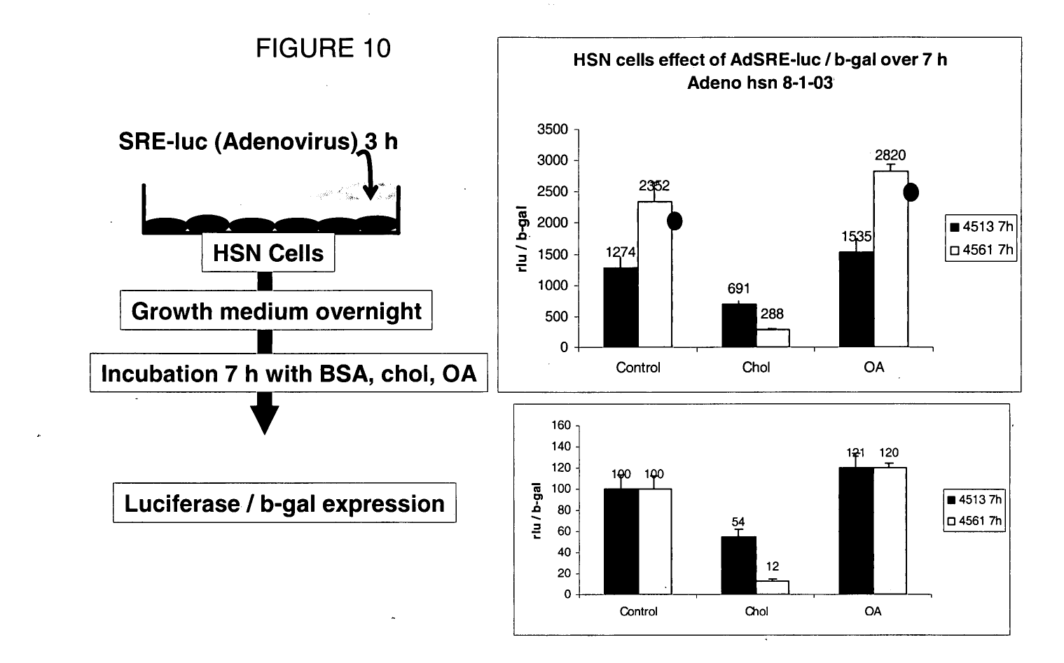

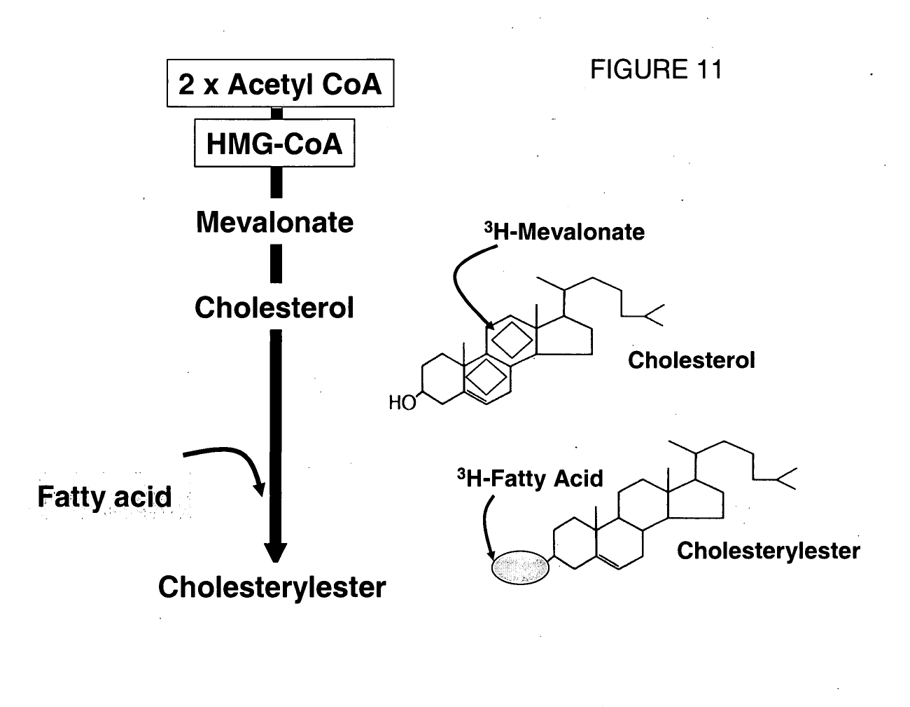

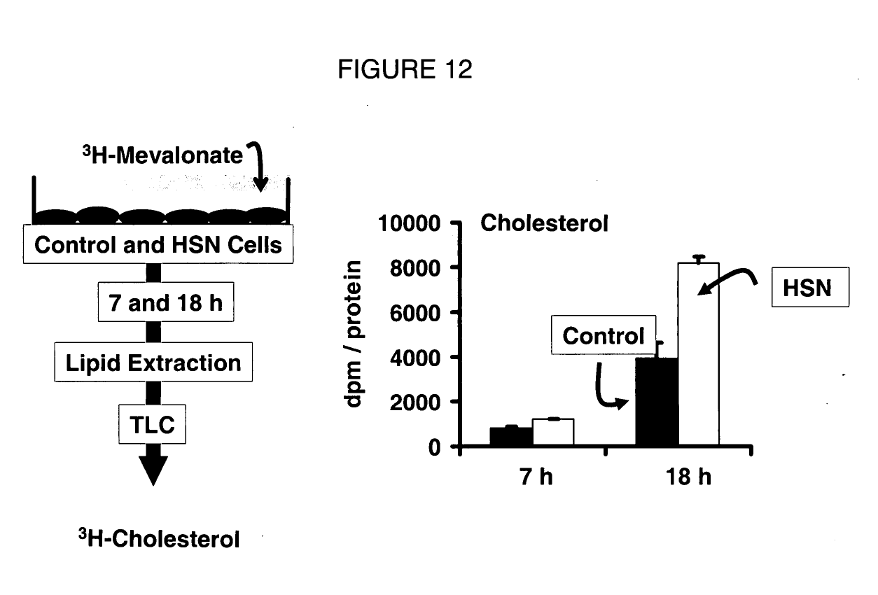

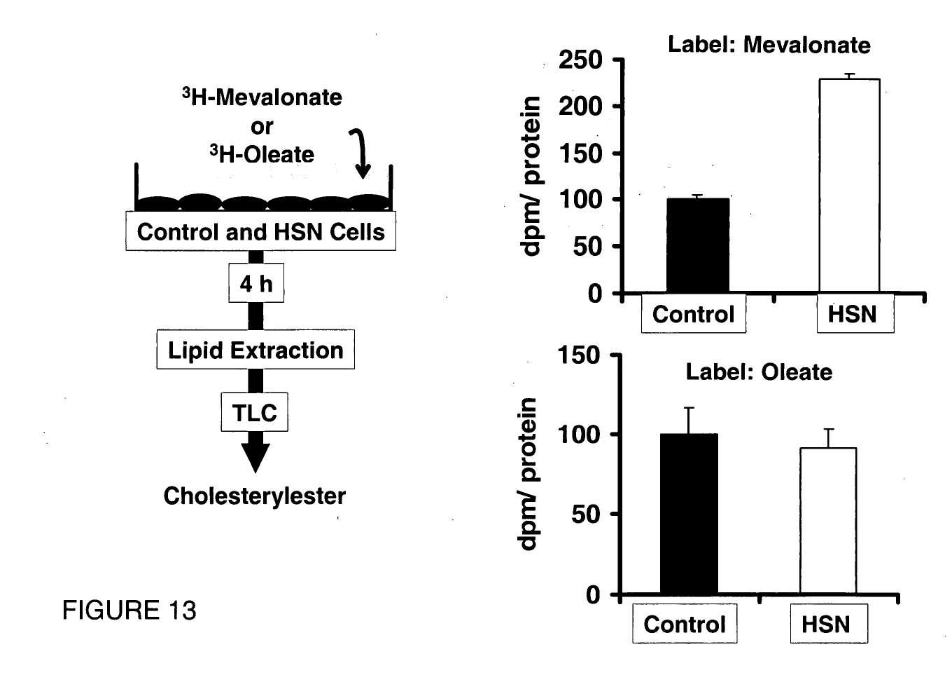

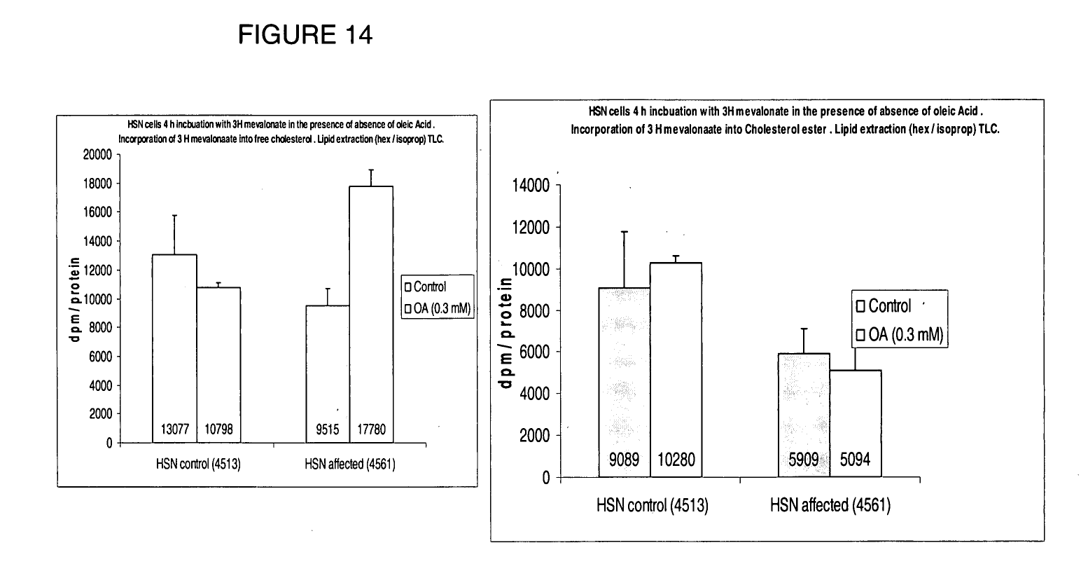

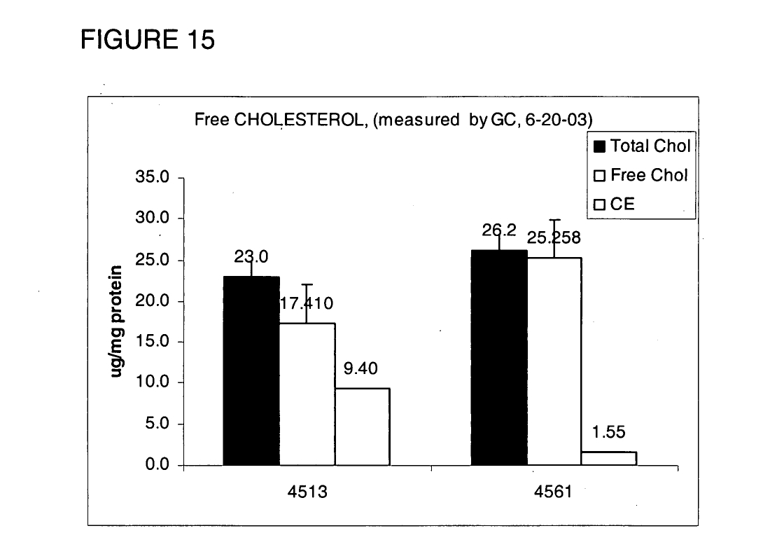

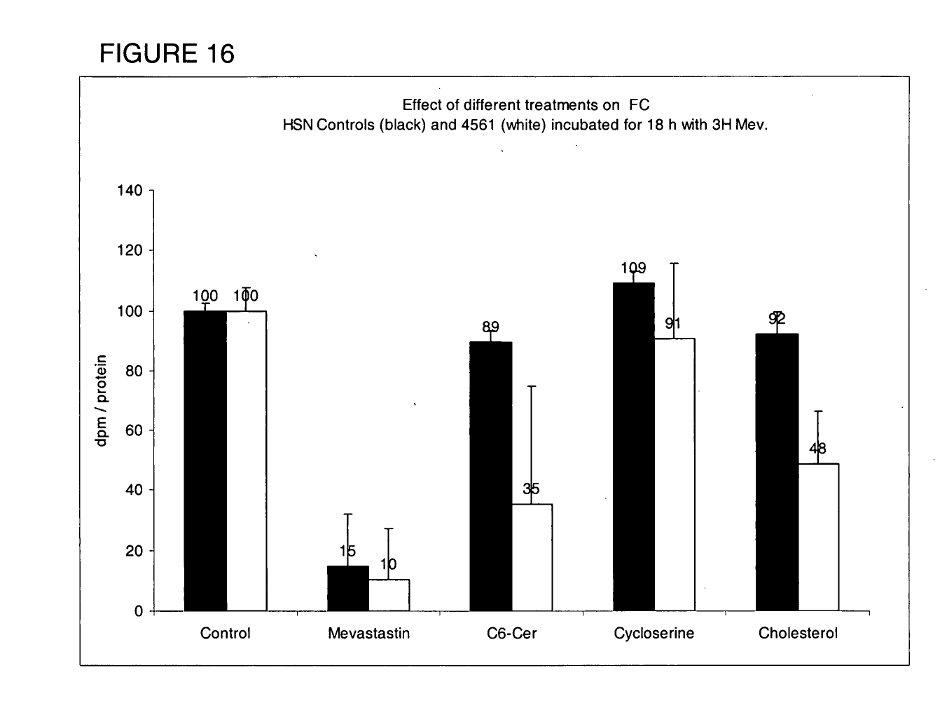

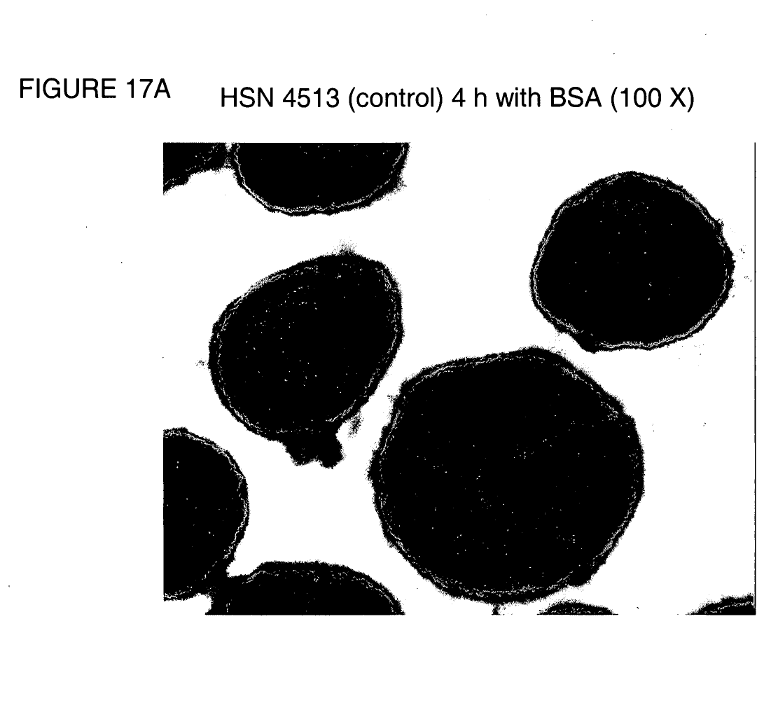

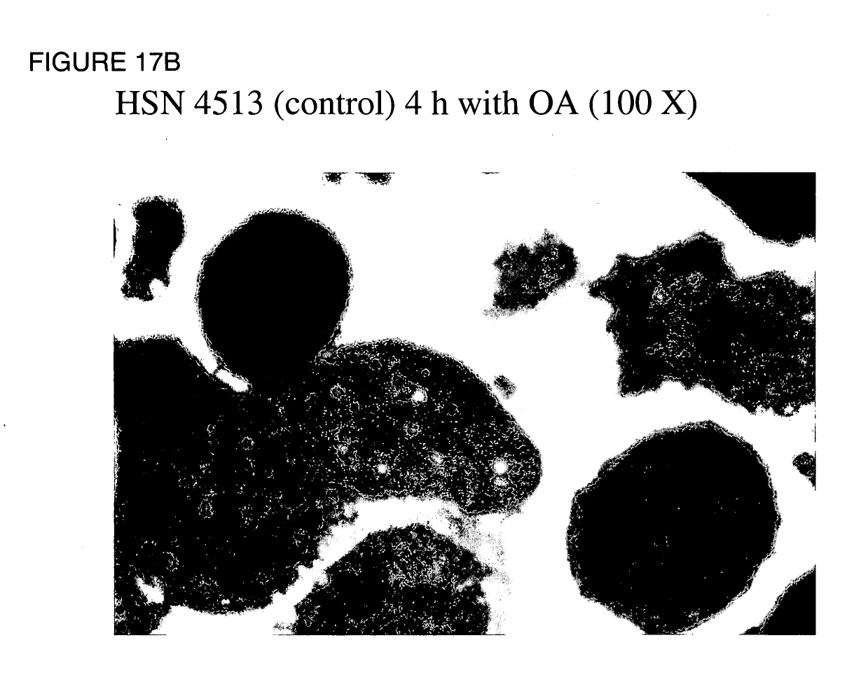

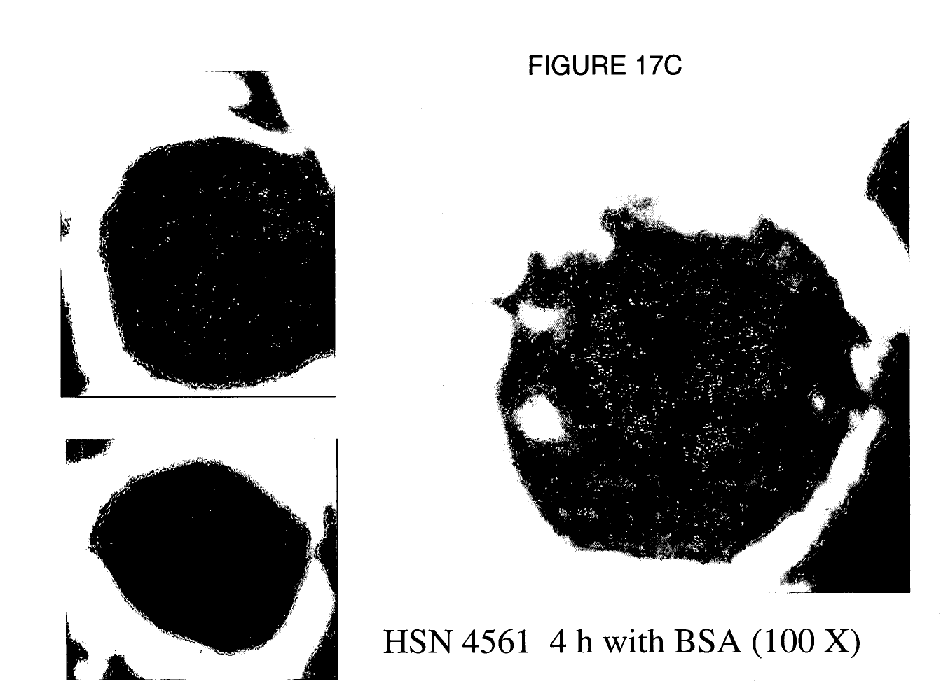

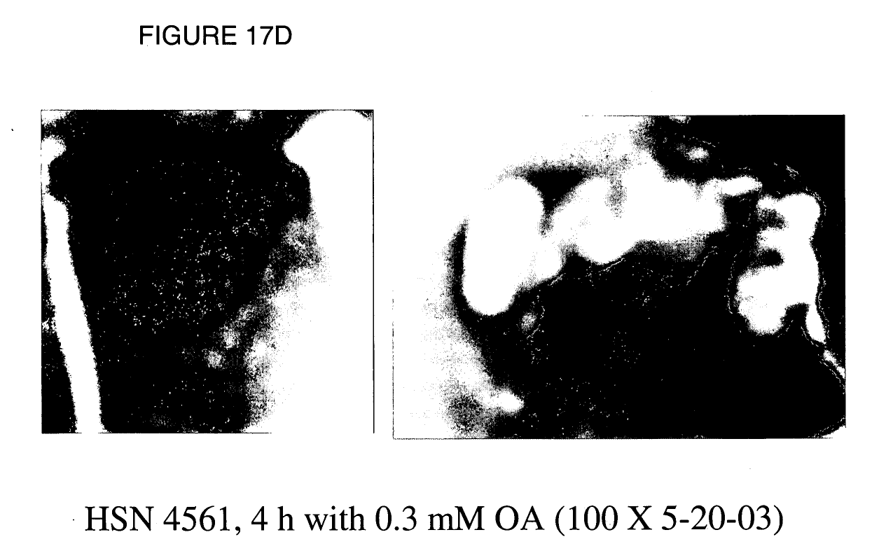

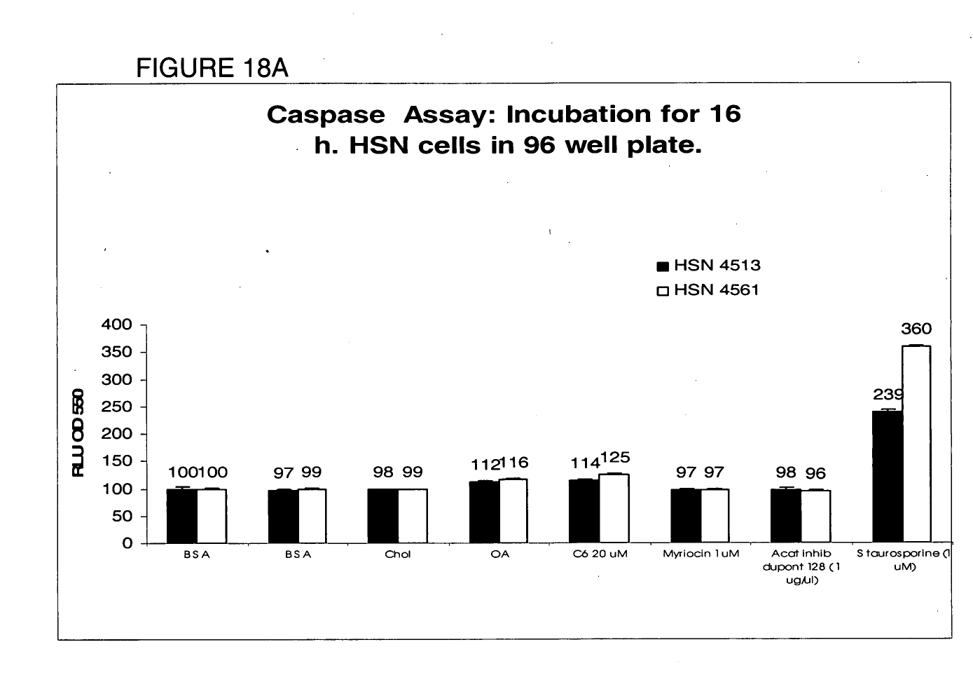

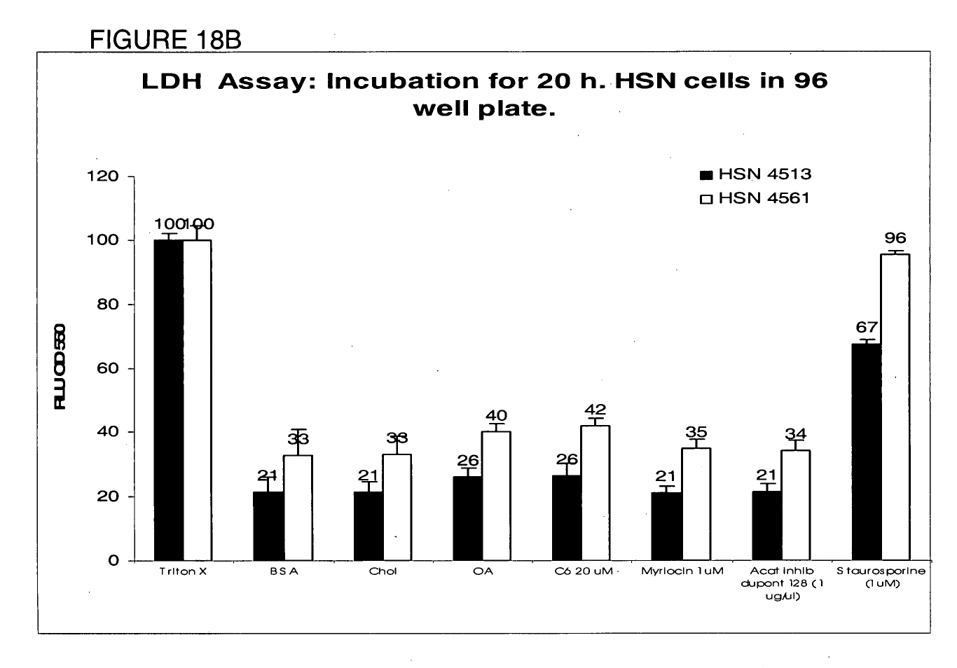

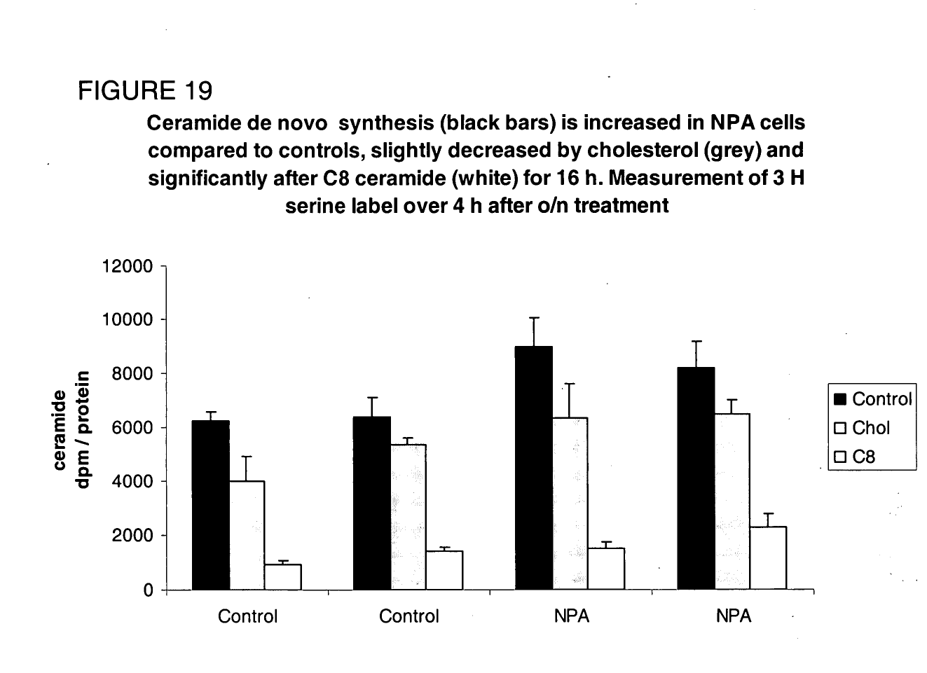

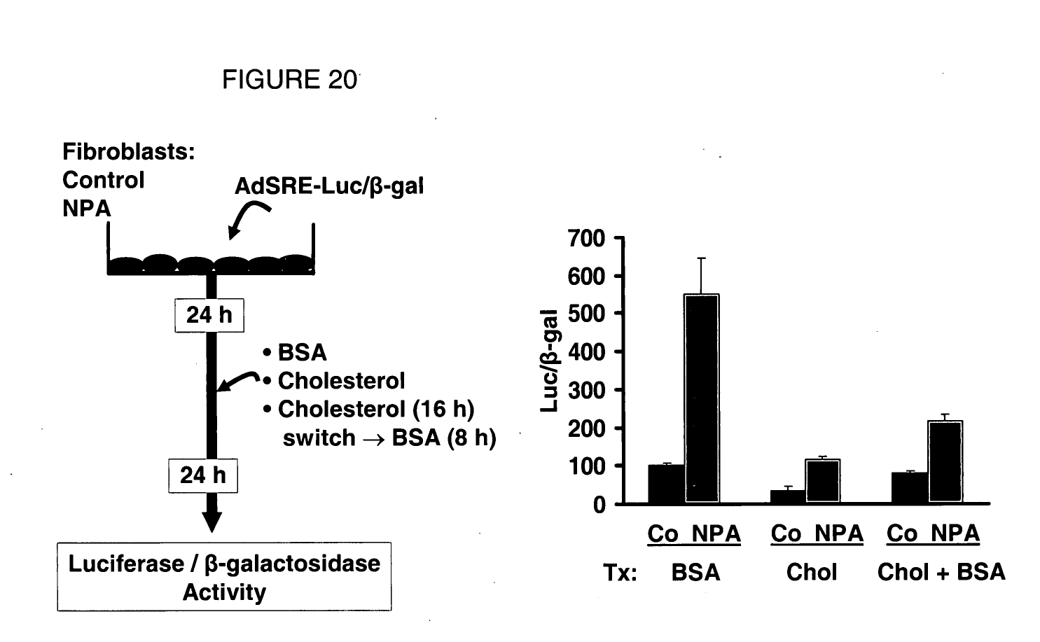

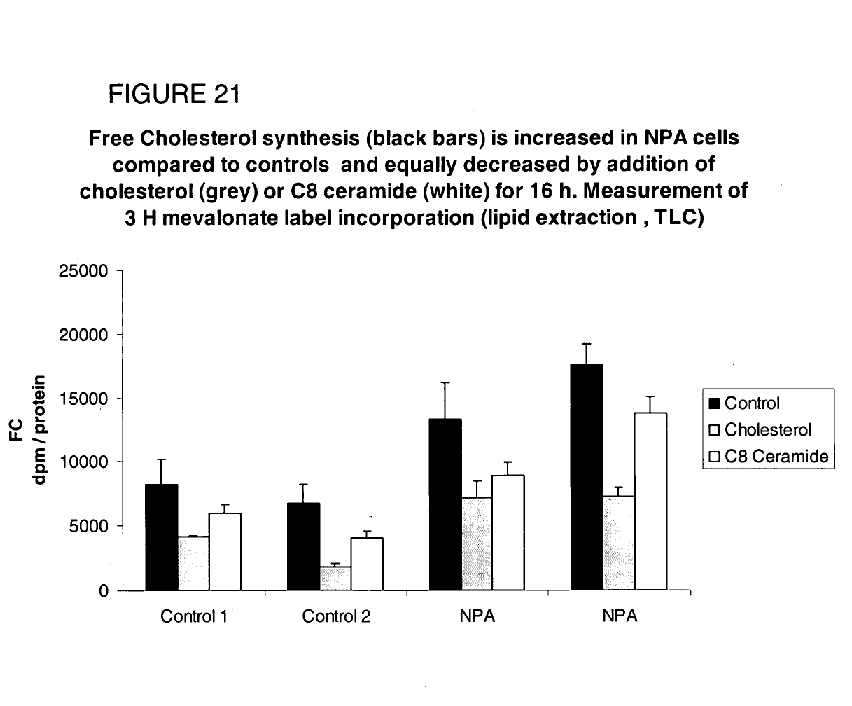

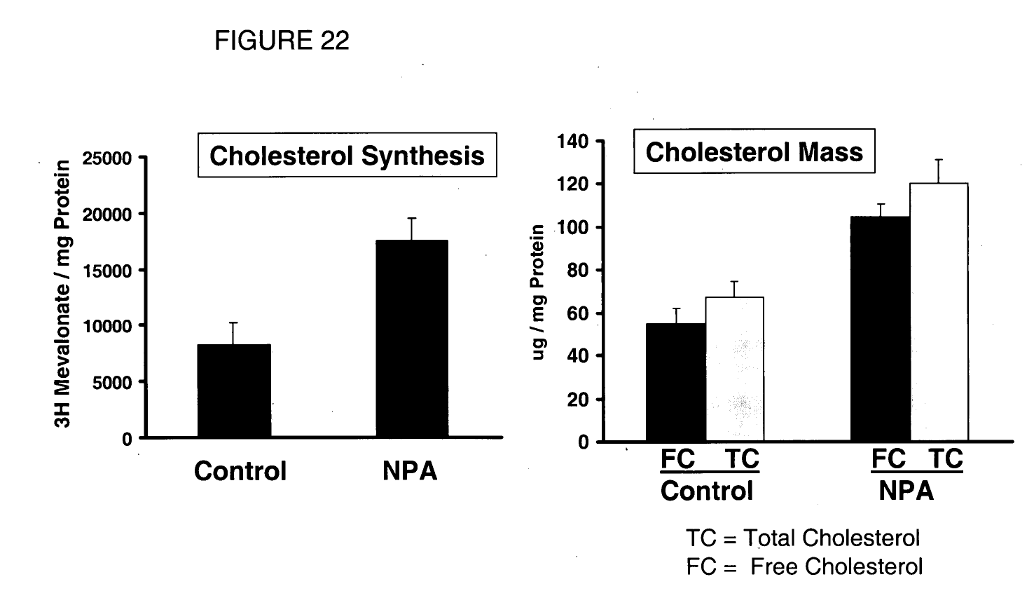

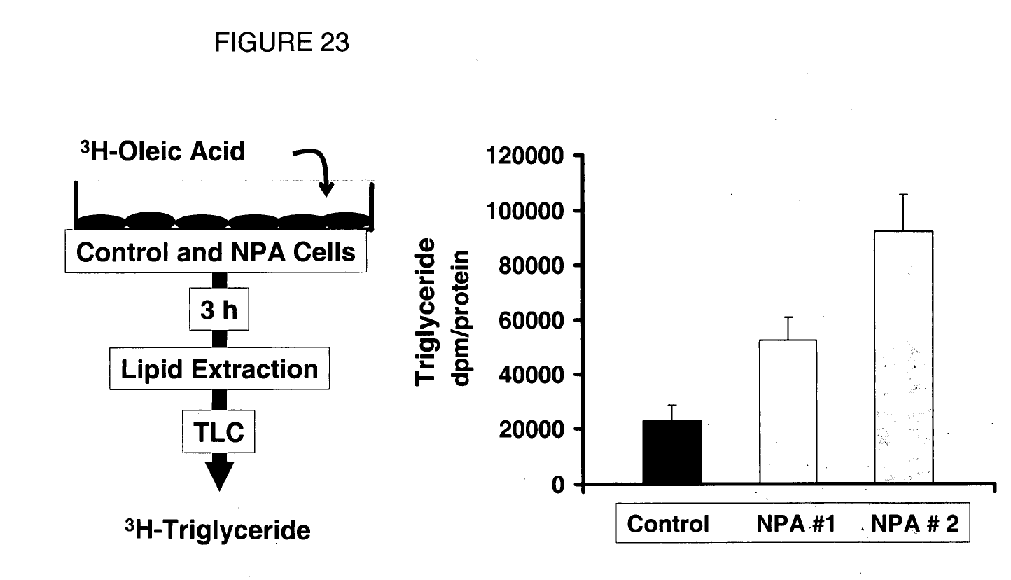

Pathways that inhibit the generation of diacylglycerol—effect on SRE-mediated gene transcription: Background (see FIGS. 7 and 8 for pathways): Sphingomyelin synthase catalyzes the reaction that transfers the phosphocholine headgroup of phosphatidylcholine (PC) to ceramide resulting in the production of sphingomyelin (SM). This transfer produces diacylglycerol (DAG) from PC. The generation of DAG is inhibited by fumonisin. Another reaction that results in the formation of diacylglyercol occurs by activity of phospholipase D (PLD) and phosphatidic acid phosphatase (PAP). PLD treatment of PC generates phosphatidic acid (PA). By the action of PAP, DAG is produced from PA. PAP is inhibited by propanolol. A recent article implicates that the generation of DAG is essential for recruitment of a vesicle biogenesis factor protein kinase D (PKD) to mammalian trans-Golgi membranes in order to form a specific class of transport vesicles (6). Propanolol is an anti-adrenegic drug (β-blocker) but also inhibits phosphatidic acid phosphatase (PAP) (3, 4) and resveratrol inhibits protein kinase D (PKD) (5).

Data: Propanolol (250 mM) inhibits SRE-mediated gene transcription to 40% (+3%) within 30 min. Resvaratrol (300 mM) inhibits SRE-mediated gene transcription to 7% (±16%) within 4 h. Preliminary data in cell culture demonstrate that addition of DAG to cells attracts fluorescently labeled SREBP. These early preliminary data suggest that the generation of DAG and the activity of PKD could be an important regulating factor of intracellular trafficking of SREBP.

References and Citations to Correlate the Effect of Inhibitors in Humans

The effect of fumonisin B1 on cholesterol metabolism has been described in several animal studies. Fumonisin is a mycotoxin produced by the fungus Fusarium monoliforme, which is found in corn. Results vary, but agree on the hepatotoxicity of fumonisin B1. The cause of the hepatotoxicity is unknown and has been attributed to the increased levels of sphinganine (7, 8), which accumulates due to the inhibition of ceramide synthase. With regard to cholesterol levels: in a 20 week study of rats fed toxic levels of fumonisin, plasma cholesterol levels were significantly decreased. The authors indicate that the mechanism of the decrease in the levels of cholesterol is not clear, but could be the result of a decreased level of sphingomyelin in cell membranes that influenced cholesterol synthesis and/or metabolism (8).

The effect of myriocin on lipid metabolism has not been investigated. Myriocin is a potent immunosuppressive agent that impedes the circulation of lymphocytes (9, 10). Recently, FTY720, a compound closely related to myriocin, but without inhibitory effects on serine-palmitoyl transferase (the rate limiting step in ceramide de novo synthesis), was shown to be an agonist of sphingosine-1-phosphate. FTY720 was used in animal experiments at concentrations of 1 mg/kg. Effects on plasma lipids were not determined (11).

Animal Experiment Protocol

Background: The sterol-regulatory element binding proteins (SREBPs) are pivotal transcription factors that regulate genes of fatty acid, cholesterol and carbohydrate metabolism. SREBP-1a regulates genes of cholesterol and fatty acid metabolism. SREBP-1c mainly regulates genes of fatty acid metabolism; SREBP-2 is mainly involved in the regulation of cholesterol-related genes. SREBP is regulated transcriptionally and post-transcriptionally. Transcriptional regulation of SREBP-1c occurs through insulin and ligands to LXR (12-14) which increase levels of SREBP-1c. Polyunsaturated fatty acids decrease the transcription of SREBP-1c by antagonizing the binding of LXR to its promoter. There are three known post-transcriptional regulators: unsaturated fatty acids (15-19), oxysterols (20) and ceramide (21, 22). Applicants demonstrate that ceramide exerts its inhibitory effect on SRE-mediated gene transcription and by inhibiting ceramide de novo synthesis. The critical role of ongoing ceramide de novo synthesis is demonstrated by three lines of evidence:

Inhibition of ceramide de novo synthesis decreases SRE-mediated gene transcription. Agents that were used to decrease ceramide de novo synthesis are: (a) Ceramide analogues of different chain length that induce a negative feed-back inhibition of de novo ceramide synthesis (23), (b) Methylthiodihydroceramide, which increases the degradation of sphinganine an obligatory precursor of ceramide de novo synthesis (1) and (c) Myriocin, cycloserine and fumonisin B1, pharmacological inhibitors of ceramide de novo synthesis.

Increased ceramide de novo synthesis increases SRE-mediated gene transcription. Agents that were used to increase ceramide de novo synthesis are: (a) N,N, Dimethyl sphingosine (DMS) (dose and time dependently increases ceramide de novo synthesis) (24 and data obtained in our lab) and (b) addition of exogenous sphingosine (increases SRE-mediated gene transcription).

Cells that cannot produce ceramide de novo (i.e., LY-B (25)) fail to increase SRE-mediated gene transcription after sterol-depletion. (a) Incubation of LY-B cells after sterol depletion with DMS restores SRE-mediated gene transcription.

The mechanism of ceramide de novo synthesis mediated regulation of SRE-mediated gene transcription has been further investigated by applicants.

Background: Ceramide is the substrate for sphingomyelin synthase which converts PC and ceramide to SM. DAG is a by-product of this reaction. DAG can also be generated by PLD-mediated generation of PA and PAP. Recent evidence demonstrates that DAG in the Golgi apparatus attracts the C1a subunit of protein kinase D (PKD). Recruitment of PKD is obligatory for vesicle budding.

Preliminary data: Inhibition of PAP by propanolol or inhibition of PKD by resvaratrol both decrease SRE-mediated gene transcription. Addition of a DAG analogue that is exogenously added and thus distributes to membranes other than the Golgi results in reorientation of fluorescently labeled mature SREBP.

Significance: Increased levels of cholesterol and triglycerides are important risk factors in the development of heart disease, stroke and morbidity. Drugs commonly used in primary and secondary prevention of heart disease target enzymatic steps of cholesterol synthesis (HMG-CoA reductase inhibitors) or increase catabolism through peroxisomes by inducing peroxisomal proliferation (fibrates).

SREBP is a pivotal transcription factor that regulates genes of cholesterol and fatty acid and carbohydrate metabolism. Insulin and oxysterols can increase levels of precursor SREBP (transcriptional regulation). The precursor form is processed to the transcriptional active mature form. High levels of transcriptionally active mature SREBP increase synthesis of cholesterol and fatty acids. Our data show that inhibition of ceramide de novo synthesis decreases the generation of mSREBP. Preliminary data suggest that the generation of DAG as a product of sphingomyelin synthase could be a mechanism that regulates the generation of mSREBP from pSREBP.

Decreasing mSREBP decreases cholesterol and fatty acid synthesis. A new mechanism is described here, i.e. the inhibition of mSREBP generation through inhibition of ceramide synthesis. Drugs that reduce the generation of mSREBP present a novel mechanism of controlling plasma lipid levels and the associated morbidity.

Animals: C57 Bl/7 mice. All experiments are set up in groups of a minimum of 3 animals with mock-treated (injection of solvent) litter controls. Three different conditions are investigated: (1) a single injection; (2) a continuous infusion over 16 h; and (3) single daily injections for 2 weeks. The synthesis of cholesterol and fatty acids is determined using radioactive tracers. We use ³H-glycerol (triglyceride synthesis), ³H-mevalonate (cholesterol synthesis) and ³H-acetate (fatty acid and cholesterol synthesis). Radioactive tracers are injected 12 h before animals are sacrificed.

To investigate the effect of inhibition of ceramide de novo synthesis on SRE-mediated gene regulation, animals are treated with myriocin (1 mg/kg), ceramide (5 μmol/kg), methylthiohydroceramide (10 μmol/kg) or fumonisin (10 μmol/kg).

To investigate the effect of DAG and PKD on SRE-mediated gene regulation, animals are treated with resveratrol (300 μmol/kg) or propanolol (100 μmol/kg).

Organs to be harvested: Liver, heart, aorta, skin fibroblasts, brain, adrenals. Tissues are divided in three parts to be analyzed for protein and RNA levels and biochemical assay. For protein analysis and biochemical assays, tissues are homogenized. For Northern analysis, RNA is extracted with Trizol.

Parameters: Western blot Analysis for SREBP, Northern blot analysis for HMG-CoA synthase. Determination of free cholesterol, triglyceride, cholesterol ester mass and synthesis using established enzymatic assays and analysis of radioactive tracer incorporation. Determination of plasma lipid levels by standard enzymatic assays.

### REFERENCES

Part I and Background of Invention

1. DeBose-Boyd, R. A., Brown, M. S., Li, W. P., Nohturfft, A., Goldstein, J. L., and Espenshade, P. J. (1999) Cell 99, 703-12.
2. Nohturfft, A., Yabe, D., Goldstein, J. L., Brown, M. S., and Espenshade, P. J. (2000) Cell 102, 315-23.
3. Brown, M. S., and Goldstein, J. L. (1997) Cell 89, 331-40.
4. Worgall, T. S., Sturley, S. L., Seo, T., Osborne, T. F., and Deckelbaum, R. J. (1998) J Biol Chem 273, 25537-40.
5. Hannah, V. C., Ou, J., Luong, A., Goldstein, J. L., and Brown, M. S. (2000) J Biol Chem 276, 4365-4372.
6. Kim, H. J., Takahashi, M., and Ezaki, O. (1999) J Biol Chem 274, 25892-8.
7. Ou, J., Tu, H., Luk, A., DeBose-Boyd, R., Bashmakov, Y., Goldstein, J. L., and Brown, M. S. (2001) Proc Natl Acad Sci USA 98, 6027-6032.
8. Xu, J., Nakamura, M. T., Cho, H. P., and Clarke, S. D. (1999) J Biol Chem 274, 23577-83.
9. Seegmiller, A. C., Dobrosotskaya, I., Goldstein, J. L., Ho, Y. K., Brown, M. S., and Rawson, R. B. (2002) Dev Cell 2, 229-38.
10. Worgall, T. S., Johnson, R. A., Seo, T., Gierens, H., and Deckelbaum, R. J. (2002) J Biol Chem 277, 3878-85.
11. van Meer, G., and Holthuis, J. C. (2000) Biochim Biophys Acta 1486, 145-70.
12. Spiegel, S., and Merrill, A. H., Jr. (1996) Faseb J 10, 1388-97.
13. Gillard, B. K., Clement, R. G., and Marcus, D. M. (1998) Glycobiology 8, 885-90.
14. Pagano, R. E., Puri, V., Dominguez, M., and Marks, D. L. (2000) Traffic 1, 807-15.
15. Abousalham, A., Hobman, T. C., Dewald, J., Garbutt, M., and Brindley, D. N. (2002) Biochem J 361, 653-61.
16. Rosenwald, A. G., and Pagano, R. E. (1993) J Biol Chem 268, 4577-9.
17. Chen, C. S., Rosenwald, A. G., and Pagano, R. E. (1995) J Biol Chem 270, 13291-7.
18. Sutterlin, C., Doering, T. L., Schimmoller, F., Schroder, S., and Riezman, H. (1997) J Cell Sci 110, 2703-14.
19. Zanolari, B., Friant, S., Funato, K., Sutterlin, C., Stevenson, B. J., and Riezman, H. (2000) Embo J 19, 2824-33.
20. Horvath, A., Sutterlin, C., Manning-Krieg, U., Movva, N. R., and Riezman, H. (1994) Embo J 13, 3687-95.
21. Hanada, K., Hara, T., Fukasawa, M., Yamaji, A., Umeda, M., and Nishijima, M. (1998) J Biol Chem 273, 33787-94.
22. Fukasawa, M., Nishijima, M., Itabe, H., Takano, T., and Hanada, K. (2000) J Biol Chem 275, 34028-34034.
23. Dooley, K. A., Millinder, S., and Osborne, T. F. (1998) J Biol Chem 273, 1349-56.
24. Carmichael, J., DeGraff, W. G., Gazdar, A. F., Minna, J. D., and Mitchell, J. B. (1987) Cancer Res 47, 936-42.
25. Gallaher, W. R., Weinstein, D. B., and Blough, H. A. (1973) Biochem Biophys Res Commun 52, 1252-6.
26. Gallaher, W. R., and Blough, H. A. (1975) Arch Biochem Biophys 168, 104-14.
27. Slotte, J. P., and Bierman, E. L. (1987) Biochem J 248, 237-42.
28. Santana, P., Fanjul, L. F., and Ruiz de Galarreta, C. M. (1998) Methods in Molecular Biology 105, 217-231.
29. van Echten-Deckert, G., Klein, A., Linke, T., Heinemann, T., Weisgerber, J., and Sandhoff, K. (1997) J Lipid Res 38, 2569-79.
30. Kuksis, A., Myher, J. J., Geher, K., Shaikh, N. A., Breckenridge, W. C., Jones, G. J., and Little, J. A. (1980) J Chromatogr 182, 1-26.
31. Bartlett, R. G. (1959) J Biol Chem 234, 466-469.
32. Abe, A., Inokuchi, J., Jimbo, M., Shimeno, H., Nagamatsu, A., Shayman, J. A., Shukla, G. S., and Radin, N. S. (1992) J Biochem (Tokyo) 111, 191-6.
33. Platt, F. M., Neises, G. R., Karlsson, G. B., Dwek, R. A., and Butters, T. D. (1994) J Biol Chem 269, 27108-14.
34. Hanada, K., Nishijima, M., Fujita, T., and Kobayashi, S. (2000) Biochem Pharmacol 59, 1211-6.
35. Sundaram, K. S., and Lev, M. (1984) J Neurochem 42, 577-81.
36. Perez-Sala, D., Cerdan, S., Ballesteros, P., Ayuso, M. S., and Parrilla, R. (1986) J Biol Chem 261, 13969-72.
37. Wang, E., Norred, W. P., Bacon, C. W., Riley, R. T., and Merrill, A. H., Jr. (1991) J Biol Chem 266, 14486-90.
38. Merrill, A. H., Jr., and Wang, E. (1986) J Biol Chem 261, 3764-9.
39. Merrill, A. H., Jr., Wang, E., and Mullins, R. E. (1988) Biochemistry 27, 340-5.
40. Kobayashi, T., Shinnoh, N., and Goto, I. (1989) Eur J Biochem 186, 493-9.
41. Edsall, L. C., Van Brocklyn, J. R., Cuvillier, O., Kleuser, B., and Spiegel, S. (1998) Biochemistry 37, 12892-8.
42. Yatomi, Y., Ruan, F., Megidish, T., Toyokuni, T., Hakomori, S., and Igarashi, Y. (1996) Biochemistry 35, 626-33.
43. Wang, X., Sato, R., Brown, M. S., Hua, X., and Goldstein, J. L. (1994) Cell 77, 53-62.
44. van Echten-Deckert, G., Giannis, A., Schwarz, A., Futerman, A. H., and Sandhoff, K. (1998) J Biol Chem 273, 1184-91.
45. Ridgway, N. D., and Merriam, D. L. (1995) Biochim Biophys Acta 1256, 57-70.
46. Bielawska, A., Greenberg, M. S., Perry, D.; Jayadev, S., Shayman, J. A., McKay, C., and Hannun, Y. A. (1996) J Biol Chem 271, 12646-54.
47. Muniz, M., Morsomme, P., and Riezman, H. (2001) Cell 104, 313-20.
48. Sheng, Z., Otani, H., Brown, M. S., and Goldstein, J. L. (1995) Proc Natl Acad Sci USA 92, 935-8.
49. Nilsson, I., Ohvo-Rekila, H., Slotte, J. P., Johnson, A. E., and von Heijne, G. (2001) J Biol Chem 276, 41748-54.
50. Scheek, S., Brown, M. S., and Goldstein, J. L. (1997) Proc Natl Acad Sci USA 94, 11179-83.
51. Wang, X., Sato, R., Brown, M. S., Hua, X., and Goldstein, J. L. (1994) Cell 77, 53-62.
52. Edwards, P. A., Tabor, D., Kast, H. R., and Venkateswaran, A. (2000) Biochim Biophys Acta 1529, 103-113.
   Part II
1. van Echten-Deckert, G., Giannis, A., Schwarz, A., Futerman, A. H., and Sandhoff, K., J Biol Chem, 273, 1184 (1998).
2. Luberto, C., and Hannun, Y. A., J Biol Chem, 273, 14550 (1998).
3. Cheng, C. H., and Saggerson, E. D., FEBS Lett, 93, 120 (1978).
4. Pappu, A. S., and Hauser, G., Neurochem Res, 8, 1565 (1983).
5. Haworth, R. S., and Avkiran, M., Biochem Pharmacol, 62, 1647 (2001).
6. Baron, C. L., and Malhotra, V., Science, 295, 325 (2002).
7. Bailly, J. D., Benard, G., Jouglar, J. Y., Durand, S., and Guerre, P., Toxicology, 163, 11 (2001).
8. Liu, H., Lu, Y., Haynes, J. S., Cunnick, J. E., Murphy, P., and Hendrich, S., J Agric Food Chem, 49, 4113 (2001).
9. Yoshikawa, M., Yokokawa, Y., Okuno, Y., Yagi, N., and Murakami, N., Chem Pharm Bull (Tokyo), 43, 1647 (1995).
10. Yoshikawa, M., Yokokawa, Y., Okuno, Y., and Murakami, N., Chem Pharm Bull (Tokyo), 42, 994 (1994).
11. Mandala, S., Hajdu, R., Bergstrom, J., Quackenbush, E., Xie, J., Milligan, J., Thornton, R., Shei, G. J., Card, D., Keohane, C., Rosenbach, M., Hale, J., Lynch, C. L., Rupprecht, K., Parsons, W., and Rosen, H., Science, 296, 346 (2002).
12. Yoshikawa, T., Shimano, H., Yahagi, N., Ide, T., Amemiya-Kudo, M., Matsuzaka, T., Nakakuki, M., Tomita, S., Okazaki, H., Tamura, Y., Iizuka, Y., Ohashi, K., Takahashi, A., Sone, H., Osuga Ji, J., Gotoda, T., Ishibashi, S., and Yamada, N., J Biol Chem, 277, 1705 (2002).
13. Repa, J. J., Liang, G., Ou, J., Bashmakov, Y., Lobaccaro, J. M., Shimomura, I., Shan, B., Brown, M. S., Goldstein, J. L., and Mangelsdorf, D. J., Genes Dev, 14, 2819 (2000).
14. Liang, G., Yang, J., Horton, J. D., Hammer, R. E., Goldstein, J. L., and Brown, M. S., J Biol Chem, 277, 9520 (2002).
15. Ou, J., Tu, H., Luk, A., DeBose-Boyd, R., Bashmakov, Y., Goldstein, J. L., and Brown, M. S., Proc Natl Acad Sci USA, 98, 6027 (2001).
16. Kim, H. J., Takahashi, M., and Ezaki, O., J Biol Chem, 274, 25892 (1999).
17. Xu, J., Nakamura, M. T., Cho, H. P., and Clarke, S. D., J Biol Chem, 274, 23577 (1999).
18. Xu, J., Teran-Garcia, M., Park, J. H., Nakamura, M. T., and Clarke, S. D., J Biol Chem, 276, 9800 (2001).
19. Worgall, T. S., Sturley, S. L., Seo, T., Osborne, T. F., and Deckelbaum, R. J., J Biol Chem, 273, 25537 (1998).
20. Edwards, P. A., Tabor, D., Kast, H. R., and Venkateswaran, A., Biochim Biophys Acta, 1529, 103 (2000).
21. Scheek, S., Brown, M. S., and Goldstein, J. L., Proc Natl Acad Sci USA, 94, 11179 (1997).
22. Worgall, T. S., Johnson, R. A., Seo, T., Gierens, H., and Deckelbaum, R. J., J Biol Chem, 277, 3878 (2002).
23. Ridgway, N. D., and Merriam, D. L., Biochim Biophys Acta, 1256, 57 (1995).
24. Edsall, L. C., Van Brocklyn, J. R., Cuvillier, O., Kleuser, B., and Spiegel, S., Biochemistry, 37, 12892 (1998).
25. Hanada, K., Hara, T., Fukasawa, M., Yamaji, A., Umeda, M., and Nishijima, M., J Biol Chem, 273, 33787 (1998).
26. Dawkins, J. L., et al., Mutations in SPTLC1, encoding serine palmitoyltransferase, long chain base subunit-1, cause hereditary sensory neuropathy type I. Nat Genet, 2001. 27(3): p. 309-12.
27. Bejaoui, K., et al., SPTLC1 is mutated in hereditary sensory neuropathy, type 1. Nat Genet, 2001. 27(3): p. 261-2.
28. Bejaoui, K., et al., Hereditary sensory neuropathy type 1 mutations confer dominant negative effects on serine palmitoyltransferase, critical for sphingolipid synthesis. J Clin Invest, 2002. 110(9): p. 1301-8.

1. A method for decreasing the amount of mSREBP in a cell characterized by an elevated level of mSREBP comprising contacting the cell with an agent that specifically inhibits de novo synthesis of ceramide in the cell, thereby decreasing the amount of mSREBP in the cell.

2-4. (canceled)

5. The method of claim 1, wherein the cell is a human cell.

6. The method of claim 1, wherein the cell is a hepatocyte.

7. The method of claim 1, wherein the cell is an adipocyte.

8. The method of claim 1, wherein the agent specifically inhibits the activity of an enzyme which catalyzes part of the de novo ceramide pathway.

9. The method of claim 8, wherein the enzyme is serine-palmitoyl transferase or ceramide synthase.

10. The method of claim 1, wherein the agent inhibits the expression of an enzyme which catalyzes part of the de novo ceramide pathway.

11. The method of claim 10, wherein the enzyme is serine-palmitoyl transferase or ceramide synthase.

12. The method of claim 1, wherein the agent is selected from the group consisting of (a) myriocin; (b) cycloserine; (c) Fumonisin B1; (d) PPMP; (e) compound D609; (f) methylthiodihydroceramide; (g) propanolol; and (h) resvaratrol.

13. A method for increasing the amount of mSREBP in a cell comprising contacting the cell with an agent that specifically increases de novo synthesis of ceramide in the cell, thereby increasing the amount of mSREBP in the cell.

14. The method of claim 13, wherein the cell is a human cell.

15. The method of claim 13, wherein the cell is a hepatocyte.

16. The method of claim 13, wherein the cell is an adipocyte.

17-43. (canceled)
